(19) United States
(12) Patent Application Publication
(10) Pub. No.: US 2003/0159181 A1
(43) Pub. Date: Aug. 21, 2003

(54) Method for influencing pollen development by modifying sucrose metabolism

(76) Inventors: Frederik Bornke, Quedlinburg (DE); Uwe Sonnewald, Quedlinburg (DE)

Correspondence Address: SCHWEGMAN, LUNDBERG, WOESSNER & KLUTH, P.A., P.O. BOX 2938, MINNEAPOLIS, MN, 55402, US

(21) Appl. No.: 10/223,277

(22) Filed: Aug. 14, 2002

Related U.S. Application Data
(63) Continuation of application No. PCT/EP01/01412, filed on Feb. 9, 2001.

(30) Foreign Application Priority Data
Sep. 13, 2000 (DE) 10045113.6
Feb. 14, 2000 (DE) 10006413.2

(51) Int. Cl. A01H 1/00; C07H 21/04; C12N 9/24; C12N 15/82; C12N 5/04

(52) U.S. Cl. 800/287; 435/200; 435/69.1; 435/320.1; 435/419; 536/23.2

(57) ABSTRACT

Methods for influencing pollen development by modifying the sucrose metabolism in transgenic plant cells and plants and generating male sterile plants by sucrose depletion in pollen. An expression of a protein has enzymatic activity of a sucrose isomerase in transgenic plant cells. Nucleic acid molecules contain a DNA sequence encoding a protein having the enzymatic activity of a sucrose isomerase, wherein the DNA sequence is functionally linked with the regulatory sequences of a promoter active in plants so that the DNA sequence is expressed in anthers or pollen. The invention further relates to transgenic plants and plant cells that contain the inventive nucleic acid molecule and whose male plant and plant cells are sterile due to the expression of the DNA sequence that encodes a protein having the enzymatic activity of a sucrose isomerase. The invention also relates to harvest products and the propagation material of said transgenic plants.

### RELATED APPLICATIONS

This application is a continuation under 35 U.S.C. 111(a) of International Application No. PCT/EP01/01412 filed Feb. 9, 2001 and published in German on Aug. 16, 2001 as WO 01/59135 A1, which claimed priority from German Application No. 100 45 113.6 filed Sep. 13, 2000 and German Application No. 100 06 413.2 filed Feb. 14, 2000, which applications and publication are incorporated herein by reference.

### FIELD OF THE INVENTION

The present invention relates to a method for influencing the pollen development by modifying the sucrose metabolism in transgenic plant cells and plants. The invention especially relates to a method for generating male sterile plants wherein carbohydrates are depleted from developing pollen. The invention particularly relates to the expression of a protein having the enzymatic activity of a sucrose isomerase in transgenic plant cells. The present invention further relates to nucleic acid molecules that contain a DNA sequence which codes for a protein having the enzymatic activity of a sucrose isomerase, and wherein the DNA sequence is operatively linked to the regulatory sequences of a promoter active in plants so that the DNA sequence is expressed in anthers or pollen. The present invention also relates to transgenic plants and plant cells which contain the nucleic acid molecule according to the invention and, due to the expression of the DNA sequence that encodes a protein having the enzymatic activity of a sucrose isomerase, are male sterile, as well as harvest products and propagation material of the transgenic plants.

### BACKGROUND OF THE INVENTION

As eukaryotes, plants possess two or more copies of their genetic information per cell. Each gene is generally represented by two alleles, which can be identical in the homozygous state or different in the heterozygous state. When two selected inbreeding lines are crossed, the F1 hybrids formed in the first generation, i.e., heterozygous individuals, are frequently larger, more robust and therefore more productive than the homozygous parents, probably because their two allelic gene products a) have a lower probability of being inactivated or b) have a larger reaction width. Plant breeders have used this effect, known as heterosis or hybrid vitality, for many decades to produce hybrid species.

Such hybrid lines are bred using cytoplasmic male sterility (CMS) or self-incompatibility (SI), the two most important genetic systems for preventing self-fertilisation.

The possibility of incorporating a new gene into the genome of a plant cell using gene technological methods has, during the last few years, revealed a third possibility of producing hybrid plants, namely, the use of a synthetically male-sterile system.

Male sterility produced by genetic engineering has already been achieved by various strategies. These include, among others, the expression of a ribonuclease (RNase) from Bacillus amyloliquefaciens in the tapetum (the tapetum is the cell layer which provides the pollen cells with nutrients during their development) of tobacco anthers (Mariani C. et al. (1990) Nature 347:737-741; Mariani C. et al. (1992) Nature 357:384-387), the overexpression of the rolC gene from Agrobacterium rhizogenes in tobacco (Schmülling T. et al. (1988) EMBO J. 7:2621-2629; Schmülling T. et al. (1993) Mol. Gen Genet. 237:385-394) and in potatoes (Fladung M. (1990) Plant Breeding 104:295-305) and the expression of a glucanase, whose activity prematurely destroys the callose cell wall of the microsporocyte (Worrall D. (1992) Plant Cell 4:759-771). Other approaches for the production of male sterile plants are connected with modifying the pigment composition of the blossom based on isolating and manipulating the genes involved in the flavonoid biosynthesis. Here the inhibition of a certain step in the flavonoid synthesis is generally achieved by the anti-sense technique or the expression of additional sense constructs (see, for example van der Krol A. R. et al. (1988) Nature 333:866-869; Napoli C. (1990) Plant Cell 2:279-289; van der Meer I. M. et al. (1992) Plant Cell 4:253-262; Taylor L. B. and Jorgenson R. (1992) J. Heredity 83:11-17). These studies, of which most are concerned with the trans-inactivation of chalcone synthase, confirm the assumption that flavonoids not only contribute to the blossom or flower colour, but also play an essential role in anther and pollen development.

In another approach to produce male sterility, an externally applied pre-herbicide is converted into a herbicide by the introduced transgene. Thus, in transgenic tobacco plants which express the argE gene from E. coli under the control of the TA29 promoter, the application of the non-toxic substance N-acetyl-L-phosphinotricin during pollen development results in male sterility (Kriete et al. (1996) Plant J. 9:809-818). As a result of the activity of the argE gene, the non-toxic pre-herbicide is deacetylated and converted into the cytotoxic L-phosphinotricin.

If the hybrid plant produced is a crop plant whose seeds, fruits or blossoms, i.e. generative organs, are to be harvested, a restorer system must also be introduced so that the F1 plant is again male fertile. In the case of the afore-mentioned expression of a ribonuclease, the ribonuclease activity destroys the function of the tapetum with the consequence that the pollen is no longer viable and male sterile plants are produced. In this case, a restorer system was developed based on the expression of a ribonuclease inhibitor gene, which was isolated from the same bacterium (B. amyloliquefaciens) that expresses the ribonuclease.

However, F1 hybrid lines are of particular importance not only because of their increased vitality and yield. The seed-growing and breeding industry has also acquired major commercial importance because the farmer cannot further propagate F1 hybrid species since a segregation of the positive properties occurs in the F2 generation and plants produced from seeds of F1 hybrids have a much lower resistance and performance than the F1 hybrids. The farmer must therefore buy new seed from the seed producer for each sowing.

Although intensive research is being carried out on the production of gene technologically produced hybrid lines with improved agronomic properties and less expenditure of work (since mechanical castration becomes no longer necessary), in many cases, however, the methods hitherto available for the production of male sterile plants do not yield completely satisfactory results. In addition, plants with a considerably increased sensitivity to phytopathogens are frequently obtained which makes them extremely difficult to handle in practice. There is thus a strong need for further methods for the production of male sterile plants which do not show the disadvantages of the prior art.

### SUMMARY OF THE INVENTION

It is thus an object of the present invention to provide available new methods for influencing the pollen development and thus for the production of male sterile plants, and recombinant DNA molecules which contain a DNA sequence which can be used to manipulate pollen development and, especially here, to produce male sterile plants.

This and further objects of the invention are achieved by providing the embodiments characterised in the claims.

Surprisingly, it has now been found that genes, whose expression in the anthers leads to a modification of the sucrose metabolism and especially has the effect that the developing pollen are depleted in sucrose and other carbohydrates, are suited to the production of male sterile plants. Especially useful here are DNA sequences which code for a protein having the enzymatic activity of a sucrose isomerase.

Proteins with sucrose isomerase activity catalyse the isomerisation of the disaccharide sucrose to other disaccharides. In this case, the α1→β2-glycosidic bond between the two monosaccharide units of sucrose, namely the glycosidic bond between glucose and fructose, is converted into another glycosidic bond between two monosaccharide units. Especially, sucrose isomerases, also known as sucrose mutases, catalyse the rearrangement into an α1→6 bond and/or an α1→α1 bond. In this case, the disaccharide palatinose is formed as a result of isomerisation to an α1→6 bond whereas the disaccharide trehalulose is formed during the rearrangement to an α1→α1 bond.

Examples of organisms whose cells contain nucleic acid sequences coding for a protein having sucrose isomerase activity especially include micro-organisms of the genus Pro-taminobacter, Erwinia, Serratia, Leuconostoc, Pseudomonas, Agrobacterium, Klebsiella and Enterobacter. Here particular mention may be made of the following examples of such micro-organisms: Protaminobacter rubrum (CBS 547, 77), Erwinia rhapontici (NCPPB 1578), Serratia plymuthica (ATCC 15928), Serratia marcescens (NCIB 8285), Leuconostoc mesenteroides NRRL B-521f (ATCC 10830a), Pseudomonas mesoacidophila MX-45 (FERM 11808 or FERM BP 3619), Agrobacterium radiobacter MX-232 (FERM 12397 or FERM BP 3620), Klebsiella subspecies and Enterobacter species.

Now it was surprisingly observed that in transgenic plants, in whose anthers sucrose is converted into palatinose as a result of the expression of gene technologically introduced sucrose isomerase DNA sequences, this expression of sucrose isomerase DNA sequences leads to a male sterile phenotype.

Without wishing to be bound to a hypothesis, the following explanation is currently accepted for the phenomenon now observed. Developing pollens are supplied with assimilates (photosynthates) by specialised cells of the anthers. Carbohydrates are transported into the apoplast in the form of the disaccharide sucrose. For the uptake of sugars the pollens secrete extracellular invertases, which ensure that the hexoses glucose and fructose are produced. These monosaccharides are taken up by available hexose transporters and are metabolised. As a result of the expression of a sucrose isomerase in the anthers, palatinose, among others, is formed from sucrose. However, the disaccharide palatinose can only be cleaved by corresponding hydrolases, but not by the afore-mentioned invertases. This has the result that the pollen cannot develop, with the consequence of male sterility.

This effect can also be achieved by other measures which result in a modification of the sucrose metabolism, especially in the depletion of sucrose and utilisable hexoses and thus in an undersupply of the pollen with carbohydrates. Thus, the pollen development can be disturbed, for example by the inhibition of invertases, hexose transporters and hexokinases, which leads to the male sterile phenotype of plants transformed with corresponding nucleic acid molecules. The development of functional pollen can also be prevented by the fact that osmotically active substances are produced in the anthers or accumulate there, which leads to desiccation of the developing pollen and thus to the male sterile phenotype.

In most plants, the carbon-supply of the developing pollen is provided by sucrose, which was generated in photosynthetically active leaves and loaded into the conducting tissue (assimilate conducting tissue) of the phloem. The sucrose is secreted by tapetum cells into the apoplast, hydrolysed to glucose and fructose by apoplastic invertases and are taken up into the pollen by hexose transporters. In the cytosol of the pollen the hexoses are phosphorylated by means of hexokinases and thus made available for metabolism. The hexoses are taken up along with protons, which are pumped into the apoplast by means of ATPases. As mentioned above various approaches, which inhibit the uptake and utilisation of monosaccharides are thus possible to interrupt the carbohydrate supply of the developing pollen.

The observation that the development of pollen can be effectively inhibited by influencing the sucrose metabolism, and here especially by depleting utilisable monosaccharides, can be ideally used to produce male sterile crop plants. The basic assumption for the production of such male sterile crop plants is the availability of suitable transformation systems. Over the last two decades a broad spectrum of transformation methods has been developed and established in this field. These techniques comprise the transformation of plant cells with T-DNA using Agrobacterium tumefaciens or Agrobacterium rhizogenes as the transforming agent, diffusion of protoplasts, the direct gene transfer of isolated DNA into protoplasts, injection and electroporation of DNA into plant cells, introduction of DNA by means of biolistic methods and other possibilities.

Another prerequisite for the production of plants which express DNA sequences encoding a protein having the enzymatic activity of a sucrose isomerase in their anthers, their tapetum or their pollen and are male sterile as a result of this specific expression, is that suitable DNA sequences are available.

Such sequences from Protaminobacter rubrum, Erwinia rhapontici, Enterobacter species SZ 62 and Pseudomonas mesoacidophila MX-45 are described in PCT/EP 95/00165. Reference is hereby made to the disclosure of this patent application, both with respect to the disclosed sequences themselves as well as with reference to the identification and characterisation of these and other sucrose isomerase coding sequences from other sources.

The person skilled in the art can obtain other sucrose isomerase coding DNA sequences from the relevant literature and gene databases using suitable search profiles and computer programs for screening for homologous sequences or for sequence alignments.

The person skilled in the art himself can also identify other sucrose isomerase coding DNA sequences from other organisms by means of conventional molecular biological techniques and use these DNA sequences within the scope of the present invention. Thus, for example, the person skilled in the art can derive suitable hybridisation probes from known sucrose isomerase sequences and use these probes for screening cDNA and/or genomic libraries of the particular desired organism from which a new sucrose isomerase gene is to be isolated. Here the person skilled in the art can go back to current hybridisation, cloning and sequencing methods, which are well-known and established in every biotechnology or gene technology laboratory (see, for example Sambrook et al. (1989) Molecular Cloning: A Laboratory Manual, 2nd edition, Cold Spring Harbor Laboratory Press, Cold Spring Harbor, N.Y.). The person skilled in the art can, of course, also synthesise and use suitable oligonucleotide primers for PCR amplifications of sucrose isomerase sequences using known sequences.

The same applies to other measures which result in an undersupply of developing pollen with carbohydrates and especially in the depletion of utilisable monosaccharides and therefore in male sterile plants. Here also the various points of attack such as invertases, hexose transporters and hexokinases are well known to the person skilled in the art from the literature so that he is capable of effectively inhibiting the corresponding proteins, for example, by transfer of anti-sense or sense or cosuppression constructs and thereby interrupting the utilisation of sucrose in the anthers.

As targets, here mention can be made of, as noted above: cell wall-bound invertases: here a plurality of genes or cDNA clones can be obtained from the relevant databases and publications, which allow the person skilled in the art to produce suitable constructs for inhibiting cell wall-bound invertase and to transfer them to plant cells using routine methods. Examples of suitable sequences are: Arabidopsis (Schwebel-Dugue et al. (1994) Plant Physiol. 104, 809-810), carrot (Ramloch-Lorenz et al. (1993) Plant J. 4, 545-554), tobacco (Greiner et al. (1995) Plant Physiol. 108, 825-826), tomato (Ohyama et al. (1998) Genes Genet. Syst. 73, 149-157), Vicia faba (Accession No. Z35163), Pisum sativum (Accession No. Z83339), Beta vulgaris (Accession No. X81795). Further sequences can easily be identified by homology comparisons so that inhibition of the invertases is possible in all relevant crop plants.

Besides the inhibition by anti-sense or sense constructs, invertase activity can also be suppressed by expression of invertase inhibitors. The invertase inhibitor from tobacco is given as example (see Greiner et al. (1998) Plant Physiol. 116, 733-742). Overexpression of an invertase inhibitor in transgenic plants resulted in inhibition of endogenous invertase activity in potato tubers (Greiner et al. (1999) Nat. BioTech. 17, 708-711).

Another target are hexose transporters (monosaccharide transporters). Here also a plurality of genes or cDNA clones can be obtained from the databases and publications, which allow the person skilled in the art to create constructs for the inhibition of pollen-expressed hexose transporters. Examples of published sequences are: petunia (Ylstra et al. (1998) Plant Physiol. 118, 297-304), Arabidopsis (Truernit et al. (1999) Plant J. 17, 191-201), tobacco (Sauer and Stadler (1993) Plant J. 4, 601-610), Medicago sativa (Accession No. AJ248339), Ricinus communis (Accession No. L08191). Other sequences can easily be identified by homology comparisons so that inhibition of hexose transporters is possible in all relevant crop plants. At this point it should be noted that the carbohydrates that are required for the supply of the growing pollen tube during fertilisation must also be transported (taken up) via a hexose transporter. This means that the inhibition of the transporter impedes both the pollen formation and the vitality of the pollen.

Undersupply of pollen with carbohydrates can also be achieved by inhibition of proton ATPases. Here also a plurality of genes or cDNA clones can be obtained from the databases and publications, which allow the person skilled in the art to create constructs for the inhibition of the plasma membrane-bound proton ATPase. Examples of published sequences include: Vicia faba (Nakajima et al. (1995) Plant Cell Physiol. 36, 919-924), potato (Harms et al. (1994) Plant Mol. Biol. 26, 979-988), rice (Ookura et al. (1994) Plant Cell Physiol. 35, 1251-1256). Other sequences can easily be identified by homology comparisons so that inhibition of the plasma-membrane-bound proton ATPase is possible in all relevant crop plants.

Another approach relates to the afore-mentioned hexokinases. Here, the same applies as to the other targets a plurality of genes or cDNA clones can be obtained from the databases and publications which allow the person skilled in the art to create constructs for the inhibition of hexokinase. Examples of published sequences are: spinach (Wiese et al. (1999) FEBS Lett. 461, 13-18), potato (Veramendi et al. (1999) Plant Physiol. 121-134), Brassica napus (Accession No. A1352726), Capsicum annum (Accession No. AA840716), Arabidopsis (Accession No. U28215), other sequences are easy to identify by homology comparisons so that an inhibition of the hexokinase is possible is all relevant crop plants.

In addition to sense and anti-sense constructs, inhibitors of the appropriate proteins could also be used. Examples for this would be the overexpression of invertase inhibitors (Greiner et al. (1998) Plant Physiol. 116, 733-742) or of antibodies which are directed against particular proteins. Examples of the successful expression of antibodies in plants are summarised by Whitelam and Cockburn (Trends in Plant Science (1996), 8, 268-272) and other examples can be obtained from the literature in the art.

Other approaches involve controlling the sucrose isomerase activity. As described above, the sucrose isomerase activity results in the formation of palatinose which leads to an undersupply of the relevant cells with carbohydrates. In order to avoid losses of growth, the sucrose isomerase will therefore be expressed preferably cell-specifically in the target cells. Alternatively the sucrose isomerase activity can be controlled by the expression of inhibitors. Inhibitors have been developed in nature for enzymes comparable with sucrose isomerase. An example has already been mentioned, the invertase inhibitors. Other examples are: proteinase inhibitors (e.g. in Gruden et al. (1997) Plant Mol. Biol. 34, 317-323), polygalacturonase inhibitors (e.g. in Mahalingam et al. (1999) Plant Microb. Interact. 12, 490-498) and amylase inhibitors (e.g. in Grossi et al. (1997) Planta 203, 295-303). All these inhibitors bind to the target protein and prevent its catalytic activity. Furthermore, the sucrose isomerase could be controlled by antibodies which bind to the isomerase and thus switch off its activity where desired.

Finally, for the implementation of the present invention only suitable regulatory sequences are required which provide for the expression of an operatively linked sucrose isomerase DNA sequence in the anthers, in the tapetum and/or in the pollen of the transformed plants. Here also the person skilled in the art can easily obtain suitable sequences from the prior art. Some promoter sequences especially suitable for the anther- or pollen-specific expression of coding sequences are described below.

These tissue- or cell-specific promoters are also preferably used for anti-sense or sense constructs to restrict modifications of the carbohydrate metabolism with the aim of achieving an undersupply of pollen also to the relevant tissue.

Finally, the production of chimeric gene constructs in which sucrose isomerase coding DNA sequences are under the control of regulatory sequences, which ensure an anther/tapetum/pollen-specific expression, is carried out by means of conventional cloning methods (see, for example Sambrook et al. (1989), supra).

### BRIEF DESCRIPTION OF THE DRAWINGS

FIG. 1 depicts the cloning of the amplified sucrose isomerase fragment into the vector pCR-Blunt (Invitrogen) to obtain plasmids pCR-SucIso1 (with translation start; SEQ ID NO:12) or pCR-SucIso2 (without translation start; SEQ ID NO:10).

FIG. 2 depicts plasmid pCR-PalQ which was constructed by the cloning of a palatinase sequence from E. rhapontici (fragment A, which extends from nucleotide 2-1656 of the palatinase gene (see SEQ ID NO: 1)) into the vector pCR-Blunt (Invitrogen).

FIG. 3 depicts plasmid p35S-cwIso (35S=35S promoter, cw=cell wall, Iso=sucrose isomerase). A DNA sequence that codes for a sucrose isomerase was isolated from the plasmid pCR SucIso2 (FIG. 1) by digestion with BamHI and SalI and ligated in a BamHI/SalI linearised pMA vector. Fragment A contains the 35S promoter of the Cauliflower Mosaic Virus (CaMV). Fragment B contains a proteinase inhibitor II gene from potato which is fused via a linker with the sequence ACC GAA TTG GG (SEQ ID NO:16) to the sucrose isomerase gene from Erwinia rhapontici, which comprises the nucleotides 109-1803. Fragment C contains the polyadenylation signal of gene 3 of the T-DNA of the Ti plasmid pTiACH5.

FIG. 4 depicts plasmid pMAL-SucIso. A DNA sequence that codes for a sucrose isomerase was isolated from the plasmid pCR-SucIso2 (FIG. 1) via restriction enzymes BamHI and SalI and ligated in a BamHI/SalI linearised pMAL-c2 vector (New England Biolabs). Fragment A contains a tac-promoter that allows IPTG-inducible gene expression. Fragment B contains a region of the malE gene and the initiation of translation. Fragment C contains the coding region of the sucrose isomerase. Fragment D contains the rrnB-terminator from E. coli.

FIG. 5. The DNA sequence which codes for a palatinase was fused to a leader peptide of a plant gene necessary for the transport into the endoplasmic reticulum (proteinase-inhibitor II gene from potato) and was brought under the control of the promoter from the TA29 gene in tobacco (resulting in plasmid pTA29-cwPalQ (TA29=promoter of the TA29 gene from tobacco, cw=cell wall, PalQ-palatinase) or under control of the 35S RNA promoter (resulting in plasmid p35S-cwpalQ (35S=35S RNA promoter of the CaMV, cw=cell wall, PalQ=palatinase). Fragment A contains the TA29 promoter from Nicotiana tabacum in plasmid pTA29-cwPalQ or the 35S RNA promoter of the Cauliflower Mosaic Virus in plasmid p35S-cwPalQ. Fragment B contains the nucleotides 923-1059 of a proteinase inhibitor II gene from potato which are fused via a linker with the sequence ACC GAA TTG GG (SEQ ID NO:16) to the palatinase gene from Erwinia rhapontici, which comprises the nucleotides 2-1656. Fragment C contains the polyadenylation signal of gene 3 of the T-DNA of the Ti plasmid pTiACH5), nucleotides 11749-11939. The fragments were cloned into plasmid pBIN19.

FIG. 6 depicts plasmid pCR-PalZ. Fragment A, which contains the sequence of the gene encoding trehalulase from E. rhapontici (from nucleotide 4-1659), was cloned into vector pCR-Blunt (Invitrogen).

### DETAILED DESCRIPTION

The present invention thus relates to a recombinant nucleic acid molecule comprising

a) regulatory sequences of a promoter active in anthers, in the tapetum and/or in pollen;

b) operatively linked thereto a DNA sequence which encodes a protein having the enzymatic activity of a sucrose isomerase; and

c) operatively linked thereto regulatory sequences which can serve as transcription, termination and/or polyadenylation signals in plant cells.

In connection with the present invention, a protein having the enzymatic activity of a sucrose isomerase is understood as a protein which catalyses the isomerisation of sucrose to other disaccharides, wherein the α1 →β2 glycosidic bond between glucose and fructose in the sucrose is converted into another glycosidic bond between two monosaccharide units, especially into an α1→β6 bond and/or an a1→α1 bond. Especially preferably a protein having the enzymatic activity of a sucrose isomerase will be understood as a protein being capable of isomerising sucrose to palatinose and/or trehalulose. In this case, the proportion of palatinose and trehalulose among the total disaccharides formed by isomerisation of sucrose is ≧2%, preferably ≧20%, more preferably ≧50% and most preferably ≧60%.

The DNA sequence, which encodes a protein having the enzymatic activity of a sucrose isomerase can be isolated from natural sources or synthesised by known methods. It is possible to prepare or produce desired constructs for the transformation of plants by means of current molecular biological techniques (see for example, Sambrook et al. (1989), supra). The cloning, mutagenisation, sequence analysis, restriction analysis and other biochemical and molecular biological methods usually used for gene technological manipulation in prokaryotic cells are well known to the person skilled in the art. Thus, it is not only possible to produce suitable chimeric gene constructs with the desired fusion of promoter and sucrose isomerase DNA sequence, but rather the person skilled in the art can, if desired, introduce various types of mutations into the sucrose isomerase coding DNA sequence, which results in the synthesis of proteins possibly having modified biological properties. By this means it is firstly possible to produce deletion mutants with which the synthesis of suitably truncated proteins can be achieved by progressive deletion from the 5′ or 3′ end of the coding DNA sequence. Further, it is possible to specifically produce enzymes, which are localised in specific compartments of the plant cell due to addition of suitable signal sequences. The introduction of point mutations is also very likely at positions where a modification of the amino acid sequence has an influence, for example, on the enzyme activity or the enzyme regulation. In this way it is possible to produce for example mutants that are no longer subject to the regulation mechanisms normally prevailing in the cell via allosteric regulation or covalent modification. Furthermore, mutants having a modified substrate- or product specificity can be produced. Further, mutants having a modified activity-, temperature- and/or pH-profile can be produced. The production of mutants, which have the aim to modify the enzymatic activity, preferably to yield an increase of the sucrose affinity by reducing the Km value is preferred.

In a preferred embodiment the DNA sequence, which codes for a protein having the enzymatic activity of a sucrose isomerase is selected from the group consisting of

a) DNA sequences comprising a nucleotide sequence which encode the amino acid sequence given in SEQ ID NO. 6 or fragments thereof,

b) DNA sequences which comprise the nucleotide sequence given in SEQ ID NO. 4 or parts thereof,

c) DNA sequences comprising a nucleotide sequence which hybridises with a complementary strand of the nucleotide sequence of a) or b), or parts of this nucleotide sequence,

d) DNA sequences comprising a nucleotide sequence which is degenerate to a nucleotide sequence of c), or parts of this nucleotide sequence,

e) DNA sequences which represent a derivative, analogue or fragment of a nucleotide sequence of a), b), c) or d).

Apart from the sucrose isomerase sequence from Erwinia rhapontici given in SEQ ID NO. 4, those having a particularly high affinity to sucrose, i.e. corresponding to a low Km value, are used as preferred DNA sequences, e.g. the sucrose isomerase from Pseudomonas mesacidophila (Km for sucrose 19.2 mM, Nagai et al. (1994) Biosci. Biotech. Biochem. 58:1789-1793) or Serratia plymuthica (Km for sucrose 63.5 mM; McAllister et al. (1990) Biotechnol. Lett. 12:667-672).

Within the framework of this invention the term “hybridisation” means hybridisation under conventional hybridisation conditions, preferably under stringent conditions, as described for example, in Sambrook et al. (1989, supra).

DNA sequences which hybridise with DNA sequences coding for a protein having the enzymatic activity of a sucrose isomerase may, for example be isolated from genomic or cDNA libraries. Such DNA sequences can be identified and isolated, for example, by using DNA sequences which exactly or substantially have one of the afore-mentioned sucrose isomerase coding nucleotide sequences of the prior art or parts of these sequences or the reverse complements of these DNA sequences, e.g. by hybridisation according to standard methods (see, for example, Sambrook et al. (1989), supra). Fragments used as a hybridisation probe can also be synthetic fragments produced using conventional synthesis techniques and whose sequence is substantially identical to one of the afore-mentioned DNA sequences for sucrose isomerase or a part of one of these sequences. The DNA sequences, which encode a protein having the enzymatic activity of a sucrose isomerase also comprise DNA sequences whose nucleotide sequences are degenerate to one of the DNA sequences as described above. The degeneration of the genetic code offers one skilled in the art, among other things, the possibility of adapting the nucleotide sequence of the DNA sequence to the codon preference (codon usage) of the target plant, i.e. the male sterile plant as a result of the specific expression of the sucrose isomerase DNA sequence, and thereby optimising the expression.

The above-mentioned DNA sequences also comprise fragments, derivatives and allelic variants of the DNA sequences as described above which code for a protein having the enzymatic activity of a sucrose isomerase. “Fragments” are to be understood as parts of the DNA sequence that are long enough to encode one of the proteins described. The term “derivative” in this context means that the sequences differ from the DNA sequences described above at one or several position/s but have a high degree in homology to these sequences. Homology means herein a sequence identity of at least 40 percent, especially an identity of at least 60 percent, preferably more than 80 percent and more preferably more than 90 percent. The variations to the above described DNA sequences may be caused for example by deletion, substitution, insertion or recombination.

The variations to the above mentioned DNA sequences can be caused for example by deletion, substitution, insertion or recombination.

The DNA sequences that are homologous to the above-mentioned sequences and represent derivatives of these sequences are generally variations of these sequences, which represent modifications having the same biological function. These variations can be both naturally occurring variations, for example sequences from other organisms, or mutations, wherein these mutations can have occurred naturally or have been introduced by targeted mutagenesis. Moreover, the variations can further comprise synthetic sequences. The allelic variants can be naturally occurring and synthetic variants or variants created by recombinant DNA techniques.

In a more preferred embodiment the described DNA sequence coding for a sucrose isomerase originates from Erwinia rhapontici (as given in SEQ ID No. 4).

The present invention also relates to a recombinant nucleic acid molecule comprising

a) regulatory sequences of a promoter active in anthers, in the tapetum and/or in pollen;

b) a DNA sequence linked thereto in sense or anti-sense orientation, whose transcription results in an inhibition of the plant's innate invertase, hexose transporter, hexokinase and/or proton ATPase expression, and

c) operatively linked thereto regulatory sequences which can serve as transcription, termination and/or polyadenylation signals in plant cells.

For the expression of the DNA sequence contained in the recombinant DNA molecules according to the invention in plant cells, the DNA sequence is linked to regulatory sequences, which ensure the transcription in plant cells. Any promoter active in plant cells comes into consideration here. Since according to the invention the sucrose isomerase must be expressed in anther, tapetum and/or pollen tissue, any promoter, which ensures the expression in anthers, tapetum or pollen, whether it is inter alia in anthers, tapetum or pollen or exclusively in these tissues, comes into consideration here.

The promoter can be selected so that the expression takes place constitutively or only in anther-, tapetum- and/or pollen-specific tissue, at a particular time in the plant development and/or at a time determined by external influences, biotic or abiotic stimuli (induced gene expression). With reference to the plant to be transformed, the promoter can be homologous or heterologous. When a constitutive promoter is used, a cell- or tissue-specific expression can also be achieved by inhibiting the gene expression in the cells or tissues in which it is not desired, for example, by the expression of antibodies that bind to the gene product and thus suppress its enzymatic activity, or by suitable inhibitors.

Particularly suited promoters within the teaching of the invention are anther-, tapetum- and/or pollen-specific promoters. Examples of this are:

the promoter of the tap 1 gene from Antirrhinum majus (Sommer et al. (1990) EMBO J. 9:605-613; Sommer et al. (1991) Development, Suppl. 1: 169-176; e.g. as 2,2 kb EcoRI/BamHI-restriction fragment);

the promoter of the TA29 gene (Mariani et al. (1990) Nature 347:737-741; Seurinck et al. (1990) Nucl. Acids Res. 18:3403; Gene bank Accession No. X52283);

the promoter of the RA8 gene from Oryza sativa L. (Jeon et al. (1999) Plant Mol. Biol. 39:35-44, this publication describes expression studies with RA8 promoter/GUS constructs in transgenic rice plants);

the promoter of the Bp 19 gene from Brassica napus (Albani et al. (1991) Plant Mol. Biol. 16:501-513);

the promoters of the LAT52 und LAT56 gene from tomato (Twell et al. (1990) Development 109:705-713);

the promoter of the BNA215-6 gene from Brassica campestris L. ssp. Pekinensis (Kim et al. (1997) Mol. Cells 7:21-27, promoter/GUS expression analyses in transgenic tobacco plants are described here);

the promoter of the NeIF-4A8 gene from Nicotiana tabacum (Brander and Kuhlemeier (1995) Plant Mol. Biol. 27:637-649, promoter/GUS expression studies are described here);

the promoter of the Bgp1 gene from Brassica campestris (Xu et al. (1993) Mol. Gen. Genet. 239:58-65, deletion constructs and their analysis in transgenic Arabidopsis thaliana plants are described here);

the promoter of the APG gene from A. thaliana (Roberts et al. (1993) Plant J. 3:111-120;

the promoter of the tap2 gene from snapdragon (Nacken et al. (1991) FEBS Lett. 280:155-158, which describes the molecular analysis of this anther-specific gene);

the promoters of the chiA and chiB gene from petunia (van Tunen et al. (1990) Plant Cell 2:393-401);

the pollen-specific promoters of the Bnm 1 gene from Brassica napus (Treacy et al. (1997) Plant Mol. Biol. 34:603-611, promoter/GUS expression analyses in transgenic rape plants are described in this article);

the promoter of the Bp4 gene from Brassica napus and the promoter of the NTM9 gene from Nicotiana tabacum (Custers et al. (1997) Plant Mol. Biol. 35:689-699) are active promoters at the early stages of pollen development; here a male sterile phenotype could be generated by means of promoter/barnase fusion constructs in transgenic tobacco plants;

the pollen-specific promoter of the ZM13 gene from maize (Hamilton et al. (1998) Plant Mol. Biol. 38:663-669), the sequence ranges required for pollen-specific expression were identified by mutation analyses and described;

the pollen-specific promoter of an invertase from potato (Machray et al. (1999) “The role of invertases in plant carbohydrate partitioning and beyond”, Abstracts, Workshop University of Regensburg, Oct. 3-6, 1999; promoter/GUS expression studies are described here).

The person skilled in the art can obtain other anther-specific genes or promoters from the prior art, especially from the relevant scientific journals and gene databases. In addition, the person skilled in the art is capable of isolating other suitable promoters by routine methods. Thus, the person skilled in the art can identify anther-specific regulatory nucleic acid elements using current molecular biological methods, for example, hybridisation experiments or DNA protein binding studies. In this case, as a first step, for example, total poly(A)⁺ RNA is isolated from the anther tissue of the desired organism from which the regulatory sequence is to be isolated, and a cDNA library is made. As a second step, cDNA clones based on poly(A)⁺ RNA molecules from a non-anther tissue are used to identify those clones from the first library by means of hybridisation whose corresponding poly(A)⁺ RNA molecules only accumulate in anther tissue. Then, these thus identified cDNAs are used to isolate promoters which have anther-specific regulatory elements. Other PCR-based methods for isolating suitable anther-specific promoters are also available to the person skilled in the art. The same applies, of course, also to pollen- or tapetum-specific promoters.

In a preferred embodiment the anther-specific promoter is the TA29 promoter from tobacco.

Also present are transcription or termination sequences that provide for correct transcription termination and can provide for addition of a poly(A) tail to the transcript to which a function in the stabilisation of transcripts is assigned. Such elements are described in the literature and are interchangeable in any order.

The invention further relates to vectors and micro-organisms which contain the nucleic acid molecules according to the invention and whose usage makes it possible to produce male sterile plants. The vectors especially include plasmids, cosmids, viruses, bacteriophages and other vectors common in gene technology. The micro-organisms are primarily bacteria, viruses, fungi, yeasts and algae.

The invention also relates to a method for producing male sterile plants comprising the following steps:

a) Production of a recombinant nucleic acid molecule that comprises the following sequences:

regulatory sequences of a promoter active in anthers, in the tapetum and/or in pollen;

operatively linked thereto a DNA sequence which codes for a protein having the enzymatic activity of a sucrose isomerase; and

operatively linked thereto regulatory sequences which can serve as transcription, termination and/or polyadenylation signals in plant cells;

b) Transfer of the nucleic acid molecule from a) to plant cells and

c) Regeneration of transgenic plants.

The invention further relates to a method for producing male sterile plants comprising the following steps:

a) Production of a recombinant nucleic acid molecule that comprises the following sequences:

regulatory sequences of a promoter active in anthers, in the tapetum and/or in pollen;

a DNA sequence linked thereto in sense or anti-sense orientation, whose transcription results in an inhibition of the plant's innate invertase, hexose transporter, hexokinase and/or proton ATPase expression, and

operatively linked thereto regulatory sequences which can serve as transcription, termination and/or polyadenylation signals in plant cells;

b) Transfer of the nucleic acid molecule from a) to plant cells and

c) Regeneration of transgenic plants.

The invention also relates to plant cells, which contain the nucleic acid molecules according to the invention, which code for a protein having the enzymatic activity of a sucrose isomerase. The invention also relates to harvest products and propagation material of transgenic plants as well as to the transgenic plants themselves, which contain a nucleic acid molecule according to the invention. The transgenic plants of the invention are male sterile as a result of the introduction and expression of a DNA sequence coding for a sucrose isomerase in the anthers.

All statements made herein with reference to recombinant nucleic acid molecules which encode a protein having the activity of a sucrose isomerase, whether it is in connection with the production of vectors, plant cells, host cells, transgenic plants and the like also apply to the other approaches described above for modifying the sucrose metabolism with the effect of undersupplying the developing pollen with carbohydrates.

In order to prepare the introduction of foreign genes into higher plants or their cells a large number of cloning vectors are available, which contain a replicating signal for E. Coli and a marker gene for selecting transformed bacterial cells. Examples of such vectors are pBR322, pUC series, M13 mp series, pACYC184 and the like. The desired sequence can be introduced into the vector at a suitable restriction site. The resulting plasmid is then used for the transformation of E. coli cells. Transformed E. coli cells are cultivated in a suitable medium and then harvested and lysed, and the plasmid is recovered. Restriction analyses, gel electrophoresis and other biochemical-molecular biological methods are generally used as analytic methods to characterise the plasmid DNA so obtained. After each manipulation the plasmid DNA can be cleaved and the thus obtained DNA fragments can be linked to other DNA sequences.

A plurality of techniques is available for introducing DNA into a plant host cell, wherein the person skilled in the art will not have any difficulties in selecting a suitable method in each case. As already mentioned, these techniques comprise the transformation of plant cells with T-DNA by use of Agrobacterium tumefaciens or Agrobacterium rhizogenes as the transforming agent, the fusion of protoplasts, the injection, electroporation, the direct gene transfer of isolated DNA into protoplasts, the introduction of DNA by means of biolistic methods as well as other possibilities which have been well-established for several years and belong to the normal repertoire of the person skilled in the art in plant molecular biology or plant biotechnology.

For the injection and electroporation of DNA into plant cells, no special requirements are imposed per se on the plasmids used. The same applies to direct gene transfer. Simple 20 plasmids such as pUC derivatives can be used for example. However, if entire plants are to be regenerated from these transformed cells, the presence of a selectable marker gene is recommended. The person skilled in the art is familiar to the current selection markers and there is no problem for him to select a suitable marker.

Depending on the method for introducing the desired gene into the plant cell, other DNA sequences may be required. If for example the Ti or Ri plasmid is used for the transformation of the plant cell, at least the right border, however more often both the right and left border of the T-DNA in the Ti or Ri plasmid, respectively, must be linked to the genes to be integrated as a flanking region. If agrobacteria are used for the transformation, the DNA to be integrated must be cloned into special plasmids and specifically either into an intermediate or into a binary vector. The intermediate vectors can be integrated into the Ti or Ri plasmid of the agrobacteria by homologous recombination due to sequences that are homologous to sequences in the T-DNA. This also contains the vir-region, which is required for T-DNA transfer. Intermediate vectors cannot replicate in agrobacteria. The intermediate vector can be transferred to Agrobacterium tumefaciens by means of a helper plasmid (conjugation). Binary vectors are able to replicate in E. coli as well as in agrobacteria. They contain a selection marker gene and a linker or polylinker framed by the right and left T-DNA border region. They can be transformed directly into the agrobacteria. The agrobacterial host cell should contain a plasmid carrying a vir-region. The vir-region is required for the transfer of the T-DNA into the plant cell. Additional T-DNA can be present. Such a transformed agrobacterial cell is used for the transformation of plant cells. The use of T-DNA for the transformation of plant cells has been studied intensively and has been sufficiently described in generally known reviews and plant transformation manuals. Plant explants can be specifically cultivated with Agrobacterium tumefaciens or Agrobacterium rhizogenes for the transfer of DNA into the plant cell. From the infected plant material (e.g. leaf pieces, stem segments, roots but also protoplasts or suspension-cultivated plant cells) whole plants may be regenerated again in a suitable medium that can contain antibiotics or biocides to select the transformed cells.

Once the introduced DNA has been integrated into the plant cell genome, it is generally stable there and is maintained in the progeny of the originally transformed cell as well. It normally contains a selection marker, which imparts the transformed plant cells resistance to a biocide or an antibiotic such as kanamycin, G 418, bleomycin, hygromycin, methotrexate, glyphosate, streptomycin, sulfonylurea, gentamycin or phosphinotricin and others. The individually selected marker should thus allow the selection of transformed cells from cells lacking the introduced DNA. Alternative markers are also suited for this purpose such as nutritive markers, screening markers (such as GFP, green fluorescent protein). Naturally, it could also be done without any selection marker, although this would involve a fairly high screening expenditure. If marker-free transgenic plants are desired, there are strategies available to the person skilled in the art, which allow subsequent removal of the marker gene, by e.g. cotransformation, sequence-specific recombinases.

Transgenic plants are regenerated from transgenic plant cells by usual regeneration methods using known media. By the use of normal methods, including molecular biological methods such as PCR, blot analyses, the plants thus obtained may then be analysed for the presence of introduced DNA which encodes a protein having the enzymatic activity of a sucrose isomerase.

The transgenic plant or the transgenic plant cells, respectively, can be any monocotyledonous or dicotyledonous plant or plant cell; preferably they are crop plants or cells of crop plants. More preferably these can be rape, cereals, sugar beet, maize, sunflower and soybean. In principle, however, any crop plant for which hybrid systems are especially useful and valuable is worthwhile for the implementation of the invention.

The invention also relates to propagation material and harvest products of plants according to the invention, for example fruits, seeds, tubers, rhizomes (rootstocks), seedlings, cuttings and the like.

The transformed cells grow within the plant in the usual way. The resulting plants can be cultivated normally. The plants differ in their phenotype from wild-type plants by the male sterile phenotype.

The specific expression of sucrose isomerase in the anthers of plants according to the invention or in plant cells according to the invention can be demonstrated and followed using conventional molecular biological and biochemical methods. These techniques are known to the person skilled in the art and he can easily select a suitable method of detection, for example a northern blot analysis to detect sucrose isomerase-specific RNA or to determine the level of accumulation of sucrose isomerase-specific RNA, a southern blot analysis to identify DNA sequences coding for sucrose isomerase or a western blot analysis to detect the sucrose isomerase protein encoded by the DNA sequences according to the present invention. Naturally, the person skilled in the art can, of course, also determine the detection of the enzymatic activity of sucrose isomerase using protocols available in the literature.

As mentioned initially, in addition to a system for producing male sterility in plants, it is also desirable to have a corresponding restorer system. In the case where male sterility is produced by anther-specific expression of DNA sequences, which encode a protein having the enzymatic activity of a sucrose isomerase, the male fertility can be restored as follows.

On the one hand, it is possible to use DNA sequences, which code for a protein having the enzymatic activity of a palatinase as restorer gene. The palatinase also known as palatinose hydrolase catalyses the cleavage of the disaccharide palatinose into the hexoses fructose and glucose. On the other hand, alternatively or additionally to the DNA sequences coding for a palatinase, nucleic acid sequences which code for a protein having the enzymatic activity of a trehalulase can be used as restorer genes. Trehalulase, also known as trehalulose hydrolase, catalyses the cleavage of the disaccharide trehalulose also into fructose and glucose.

Thus, the male sterile phenotype can be overcome or neutralised by crossing with plants, which express a protein having the enzymatic activity of a palatinase and/or a protein having the enzymatic activity of a trehalulase, and thus a complete hybrid system including restorer function can be realised.

Palatinase genes are known in the prior art. Thus, PCT/EP 95/00165 discloses the sequence of a palatinase gene from the bacterium Protaminobacter rubrum and the sequence of a palatinase gene from the bacterium Pseudomonas mesoacidophila MX-45.

Now disclosed for the first time as part of the present invention is a DNA sequence from Erwinia rhapontici, which codes for a protein having the enzymatic activity of a palatinase. This sequence is given in the appended sequence protocol in SEQ ID No. 1, and the derived amino acid sequence is given in SEQ ID NOs: 2 and 3.

Also provided for the first time, as part of the present invention, is a DNA sequence from Erwinia rhapontici, which encodes a protein having the enzymatic activity of a trehalulase. The sequence is given in the appended sequence protocol in SEQ ID NO: 7, and the derived amino acid sequence is given in SEQ ID NOS: 8 and 9.

In connection with palatinase and trehalulase sequences from other sources and the methods by which the person skilled in the art can isolate or produce such sequences, reference is made to the reasoning put forward above in connection with sucrose isomerase sequences in their full extent. The same applies to the production of recombinant nucleic acid molecules which code for a protein having the enzymatic activity of a palatinase or a protein having the enzymatic activity of a trehalulase for the production of plants and also for the transfer of such nucleic acid molecules to plant cells and the regeneration of transgenic plants. Reference is also expressly made to all the above reasoning on the hybridisation, homology, derivatives, variants and fragments, regulatory sequences etc.

The invention thus also relates to the nucleotide sequences given in SEQ ID NO: 1 and SEQ ID NO: 7, respectively, which encode a protein having the enzymatic activity of a palatinase or trehalulase and the use of nucleic acid molecules, which encode proteins having the enzymatic activity of a palatinase or trehalulase for the restoration of male fertility in transgenic plants.

The invention further relates to a method for the production of male fertile hybrid plants comprising the following steps:

a) Production of a first transgenic male sterile parent plant comprising a nucleic acid molecule which codes for a protein having the enzymatic activity of a sucrose isomerase,

b) Production of a second transgenic parent plant, comprising a nucleic acid molecule which encodes a protein having the enzymatic activity of a palatinase and/or a nucleic acid molecule which codes for a protein having the enzymatic activity of a trehalulase,

c) Crossing the first parent plant with the second parent plant to produce a hybrid plant, wherein the hybrid plant is male fertile.

The same promoters as are useful for the expression of the sucrose isomerase are, of course, also suitable for the expression of the palatinase or trehalulase gene. The palatinase or trehalulase DNA sequences can advantageously also be expressed under the control of constitutive promoters, such as for example the 35S RNA promoter of CaMV. Both the palatinase and the trehalulase enzyme activity have per se no influence on the plant cells and thus no influence on plant growth, even when expressed in all tissues of the transgenic plant.

Preferably used are those palatinase DNA sequences, which code for an enzyme with high affinity to palatinose. Accordingly, preferably used are those trehalulase DNA sequences, which code for an enzyme with high affinity to trehalulose.

As mentioned above, the provision of a restorer system, that is the re-establishment of the fertility of a transgenic male sterile plant, requires the production of a second, so-called restorer line of transgenic plants. This restorer line can thus be a line, which contains a DNA sequence that codes for a protein having the activity of a palatinase or a trehalulase. Other approaches to the restoration of fertility are also possible. Thus, the expression of a corresponding sucrose isomerase inhibitor in the restorer line can be used to restore fertility in sucrose isomerase-expressing male sterile plants. Such an inhibitor can, for example, be an antibody directed towards the sucrose isomerase or an inhibitor, as it is known for invertases, for example (Greiner et al. (1999) Nat. Biotechnol. 17:708-711).

In another approach, the restorer line can express a ribozyme directed towards the sucrose isomerase mRNA. Ribozymes can be produced in such a fashion that they possess endonuclease activity directed towards a specific mRNA (see, for example, Steinecke et al. (1992) EMBO J. 11: 1525).

In a similar approach the fertility can be restored by the expression of a corresponding anti-sense RNA. Binding of the antisense RNA to the target RNA results in inhibition of its translation (Paterson et al. (1987) Proc. Natl. Acad. Sci. USA 74:4370). In connection with the present invention, one would thus crossbred a plant which is male sterile due to the expression of the sucrose isomerase with a restorer line in which the corresponding sucrose isomerase sequences are under the control of suitable promoters in the anti-sense orientation so that anti-sense transcripts for sucrose isomerase are formed. By this means, the anther-specific sense expression, which brings about the male sterile phenotype is inhibited or neutralised so that male fertile crossing products are formed. Alternatively, the phenomenon of cosuppression can be used in the same way as the anti-sense technique to restore male fertility. In a preferred embodiment of the invention, the expression of the anti-sense or cosuppression RNA is under the control of an inducible promoter whose activation allows the specific restoration of the male fertility.

Another alternative includes the expression of a RNA transcript, which causes the RNAse-P-mediated cleavage of sucrose isomerase mRNA molecules. In this approach an external leader sequence is constructed which directs the endogenous RNAse-P to sucrose isomerase mRNA and finally mediates the cleavage of this mRNA (Altman et al., U.S. Pat. No. 5,168,053; Yuan et al. (1994) Science 263:1269). Preferably the external leader sequence includes 10 to 15 nucleotides complementary to sucrose isomerase and a 3′-NCCA nucleotide sequence wherein N is preferably a purine. The transcripts of the external leader sequence bind to the target mRNA via base pairing which facilitates the cleavage of the mRNA by the RNAse-P at the nucleotide 5′ from the base paired region.

Another approach to the restoration of male fertility are transgenic, male sterile plants which, in addition to a sucrose isomerase gene operatively linked to a promoter sequence, contain a prokaryotic control region within the same expression cassette. Transgenic male fertile plants, which express a prokaryotic polypeptide under the control of a suitable promoter, are additionally produced. In the F1 hybrids the prokaryotic polypeptide binds to the prokaryotic control region and represses the expression of the sucrose isomerase. Specifically, the LexA gene/LexA operator system can be used to control the gene expression (U.S. Pat. No. 4,833,080; Wang et al. (1993) Mol. Cell Biol. 13:1805). This would mean that the expression cassette of the male sterile line contains the LexA operator sequence whereas the expression cassette of the male fertile restorer line contains the coding region of the LexA repressor. In the F1 hybrids the LexA repressor binds to the LexA operator region and thereby prevents transcription of the sucrose isomerase gene. LexA operator-DNA molecules can be obtained, for example by the synthesis of DNA fragments, which contain LexA operator sequences well known to the person skilled in the art from the literature, as described, for example, by Garriga et al. (1992) Mol. Gen. Genet. 236:125. DNA sequences which code for the LexA repressor can, for example, be obtained by synthesis of such DNA molecules or by DNA cloning techniques as are known to the person skilled in the art and are described, for example by Garriga et al., vide supra. Alternatively, sequences coding for the LexA repressor can be taken, for example, from plasmid pRB500 (ATTC 67758).

The approach explained in connection with the LexA repressor/operator system for the re-establishment of male fertility can also be achieved with other repressor/operator systems as the person skilled in the art knows them from the literature in a plurality of ways, e.g. the Lac repressor/lac operator system or the trp repressor/trp operator system.

Finally, the fertility can be restored by application of chemical compounds or substances during pollen development, which inhibit the activity of the sucrose isomerase.

The invention is based on the successful production of new plants which are male sterile due to the introduction and expression of a nucleic acid sequence coding for a sucrose isomerase in the anthers, which is explained in the following examples which serve merely to illustrate the invention and are in no way to be understood as restrictive.

### EXAMPLES

Gene technological methods on which the embodiments are based:

1. General Cloning Methods

Cloning methods, such as for example: restriction cleavage, DNA isolation, agarose gel electrophoresis, purification of DNA fragments, transfer of nucleic acids onto nitrocellulose and nylon membranes, linking of DNA fragments, transformation of E. coli cells, cultivation of bacteria, sequence analysis of recombinant DNA, were performed as described in Sambrook et al. (1989, vide supra). The transformation of Agrobacterium tumefaciens was carried out according to the method of Höfgen and Willmitzer (1988, Nucl. Acids Res. 16:9877). Agrobacteria were cultivated in YEB medium (Vervliet et al. (1975) Gen. Virol. 26:33).

2. Production of a Genomic Library of Erwinia rhapontici

In order to produce a genomic library from Erwinia rhapontici (DSM 4484) chromosomal DNA was isolated from the cells of a 50 ml overnight culture according to a standard protocol. Approximately 300 μg of the DNA were then partially digested with the restriction enzyme Sau3A and separated on a preparative agarose gel. Fragments between 5 and 12 kb were eluted from the gel using the Qiaquick Gel Extraction Kit (Qiagen, Hilden). The resulting DNA fragments were ligated in BamHI-digested Lambda ZAP-Express-Arme (Stratagene, La Jolla, USA) and then packed in vitro (Gigapack III Gold Packaging Extract, Stratagene, according to the manufacturer's data). E. coli bacteria of the strain XL-MRF′ (Stratagene) were then infected with recombinant lambda phages, the titre of the library was determined and the library was then amplified.

3. Screening of a Genomic Library

Approximately 10⁵ phages were plated for the isolation of genomic clones. After transferring the phages onto nylon filters (Genescreen, NEN) the filters were hybridised with a radioactively labelled DNA fragment. Positive signals were visualised by autoradiography and singling out was performed.

4. Bacterial Strains and Plasmids

E. coli (XL-1 Blue, XL-MRF′ and XLOLR) bacteria were obtained from Stratagene. Erwinia rhapontici (DSM 4484) was obtained from the Deutsche Sammlung für Mikro-organismen und Zellkulturen GmbH (Braunschweig, Germany). The agrobacterial strain used for the transformation of plants (C58C1 with the plasmid pGV 3850kan) was described by Debleare et al. (1985, Nucl. Acids Res. 13:4777). The vectors pCR-Blunt (Invitrogen, Netherlands), pMAL-c2 (New England Biolabs), pUC19 (Yanish-Perron (1985) Gene 33:103-119) and Bin19 (Bevan (1984) Nucl. Acids Res. 12:8711-8720) were used for the cloning.

5. Transformation of Tobacco

For the transformation of tobacco plants (Nicotiana tabacum L. cv. Samsun NN) 10 ml of a overnight culture of Agrobacterium tumefaciens grown under selection was centrifuged, the supernatant was discarded, and the bacteria were resuspended in the same volume, but antibiotic-free medium. Leaf disks of sterile plants (diameter approx. 1 cm) were bathed in this bacteria solution in a sterile culture dish. The leaf disks were then placed into Petri dishes onto MS medium (Murashige and Skoog (1962) Physiol. Plant 15:473) containing 2% sucrose and 0.8% Bacto-agar. After incubation for 2 days in darkness at 25° C. they were transferred to MS medium containing 100 mg/l kanamycin, 500 mg/l claforan, 1 mg/l benzylaminopurine (BAP), 0.2 mg/l naphthylacetic acid (NAA), 1.6% glucose and 0.8% Bacto-agar and cultivation was continued (16 hours light/8 hours darkness). Growing shoots were transferred to a hormone-free MS medium containing 2% sucrose, 250 mg/l claforan and 0.8% Bacto-agar.

6. Detection of Palatinose in Plant Extracts

In order to detect sucrose isomerase activity in plant extracts, leaf disks having a diameter of approx. 0.8 cm were extracted for 2 h at 70° C. using 100 μl 80% ethanol and 10 mM HEPES buffer (pH 7.5). A HPLC system from Dionex that was equipped with a PA-1 (4×250 mm) column and a pulsed electrochemical detector was used to analyse an aliquot of these extracts. Prior to injection the samples were centrifuged for 2 minutes at 13,000 rpm. Sugars were then eluted using a gradient of 0 to 1 M sodium acetate for 10 minutes, after 4 minutes at 150 mM NaOH and a flow rate of 1 ml/min. Suitable standards obtained from Sigma were used to identify and quantify the sugars.

### Example 1

PCR Amplification of a Subfragment of Sucrose Isomerase from Erwinia rhapontici

A subfragment of sucrose isomerase was cloned by polymerase chain reaction (Polymerase Chain Reaction, PCR). The template material was genomic DNA from E. rhapontici (DSM 4484), which was isolated according to a standard protocol. The amplification was carried out using the specific primers

| | |
|---|---|
| FB83 | |
| 5′-GGATCCGGTACCGTTCAGCAATCAAAT-3′ | (SEQ ID NO:10) |
| and | |
| FB84 | |
| 5′-GTCGACGTCTTGCCAAAAACCTT-3′, | (SEQ ID NO:11) |

which were derived from a sucrose isomerase sequence of the prior art. Primer FB 83 comprises the bases 109-127 and primer FB 84 comprises the bases 1289-1306 of the coding region of the sucrose isomerase gene from E. rhapontici. The PCR reaction mix (100 μl) contained bacterial chromosomal DNA (1 μg), primers FB 83 and FB 84 (250 ng of each), Pfu DNA polymerase reaction buffer (10 μl, Stratagene), 200 μM dNTPs (dATP, dCTP, dGTP, dTTP) and 2.5 units of Pfu DNA polymerase (Stratagene). Prior to the initiation of the amplification cycles the mixture was heated for 5 min to 95° C. The polymerisation steps (30 cycles) were carried out in an automated T3-Thermocycler (Biometra) according to the following program: denaturation at 95° C. (1 minute), annealing of the primers at 55° C. (40 seconds), polymerase reaction at 72° C. (2 minutes). The resulting fragment was cloned into the vector pCR-Blunt (Invitrogen). The identity of the amplified DNA was verified by sequence analysis.

The amplified subfragment can well be used as a hybridisation probe for the isolation of further sucrose isomerase DNA sequences from other organisms or as a probe for the analysis of transgenic cells and plants.

### Example 2

Isolation and Sequencing of the Palatinose Operon from E. rhapontici

A genomic library was screened with a subfragment of the sucrose isomerase (see Example 1) to isolate the palatinose operon. Hence, several positive clones were isolated. By complete sequencing and linking of these clones it was possible to identify several open reading frames which code for enzymes of palatinose metabolism (see overview of the genes of the palatinose operon and the respective gene products as given below). The following draft gives a schematic overview of the cloned palatinose gene cluster from Erwinia rhapontici. Arrows indicate the position of the open reading frames and the direction of transcription.

| Gene | Function of the gene product |
|---|---|
| palI | sucrose isomerase |
| palR | regulator protein of the LysR family |
| palE | palatinose binding protein, component of the ABC-transporter |
| | system for the uptake of palatinose into the cell |
| palF | integral membrane protein, permease, component of the ABC- |
| | transporter system |
| palG | integral membrane protein, permease, component of the ABC- |
| | transporter system |
| palH | presumably hydrolase activity |
| palK | ATP binding protein, component of the ABC-transporter |
| | system, provides energy for the uptake of palatinose into the cell |
| palQ | palatinase |
| palZ | trehalulase |

### Example 3

PCR Amplification of a Sucrose Isomerase from Erwinia rhapontici

The entire open reading frame of sucrose isomerase was cloned by means of polymerase chain reaction (Polymerase Chain Reaction, PCR). The template material was genomic DNA from E. rhapontici (DSM 4484), which was isolated according to a standard protocol. The amplification was carried out using the specific primers

(SEQ ID NO:12)

| | |
|---|---|
| FB96 | 5′-GGATCCACAATGGCAACCGTTCAGCAATCAAAT-3′ |
| and | |

(SEQ ID NO:13)

| | |
|---|---|
| FB97 | 5′-GTCGACCTACGTGATTAAGTTTATA-3′. |

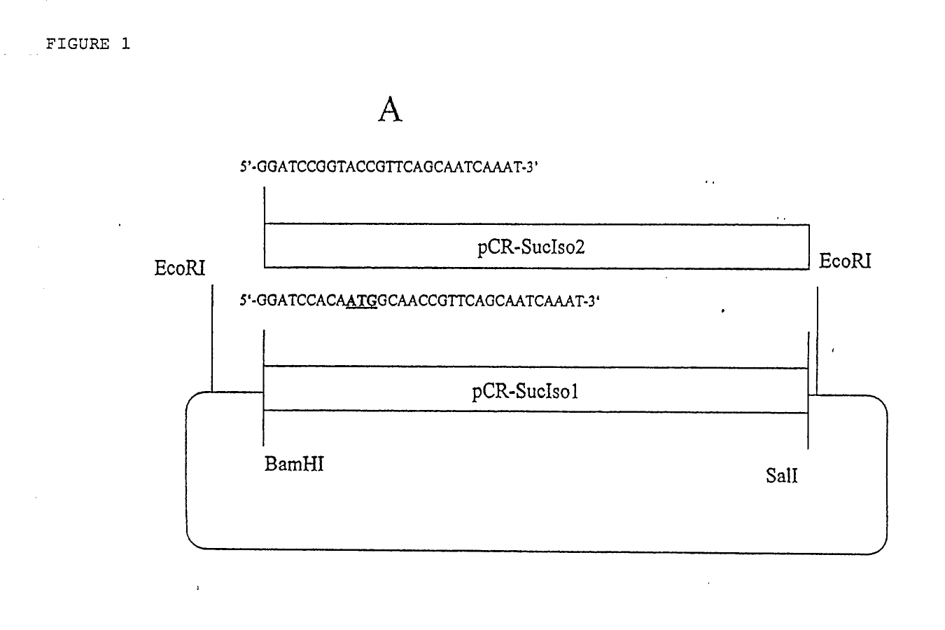

for pCR-SucIso1. Primer FB 96 comprises the bases 109-127 and additionally contains a start codon, primer FB 97 contains the bases 1786-1803 of the coding region of the sucrose isomerase gene. FB 83 (5′-GGATCCGGTACCGTTCAGCAATCAAAT-3′; SEQ ID NO:10), which contains no additional ATG, was used as 5′ primer to produce the construct pCR-SucIso2. For cloning the amplified DNA into expression vectors the primers also contain the following restriction sites: primer FB 96 or FB 83, BamHI; primer FB 97, SalI. The PCR reaction mix (100 μl) contained bacterial chromosal DNA (1 μg), primer FB 96 and FB 97 for pCR-SucIso1 or primer FB 83 and FB 97 for pCR-SucIso2 (250 ng in each case), Pfu DNA polymerase reaction buffer (10 μl, Stratagene), 200 μM dNTPs (dATP, dCTP, dGTP, dTTP) and 2.5 units of Pfu DNA polymerase (Stratagene). Prior to the initiation of the amplification cycles the mixture was heated for 5 min to 95° C. The polymerisation steps (30 cycles) were carried out in an automated T3-Thermocycler (Biometra) according to the following program: denaturation at 95° C. (1 minute), annealing of the primers at 55° C. (40 seconds), polymerase reaction at 72° C. (2 minutes). The amplified sucrose isomerase fragment was cloned into the vector pCR-Blunt (Invitrogen) by means of which the plasmid pCR-SucIso1 (with translation start) or pCR-SucIso2 (without translation start) was obtained (see FIG. 1). The identity of the amplified DNA was verified by means of sequence analysis.

The fragment A contains the sequence of a sucrose isomerase from E rhapontici, which extends from nucleotide 109-1803 of the sucrose isomerase gene. The nucleotide sequence of the primer used was underlined in each case. The DNA sequence is given in SEQ ID NO: 4.

### Example 4

PCR Amplification of a Palatinase from Erwinia rhapontici

The entire open reading frame of the palatinase was cloned using polymerase chain reaction (Polymerase Chain Reaction, PCR). The template material was genomic DNA from E. rhapontici, which was isolated according to a standard protocol. The amplification was carried out using the specific primers:

| | |
|---|---|
| FB180 | |
| 5′-GAGATCTTGCGCAGCACACCGCACTGG-3′ | (SEQ ID NO:14) |
| FB176 | |
| 5′-GTCGACTCACAGCCTCTCAATAAG-3′ | (SEQ ID NO:15) |

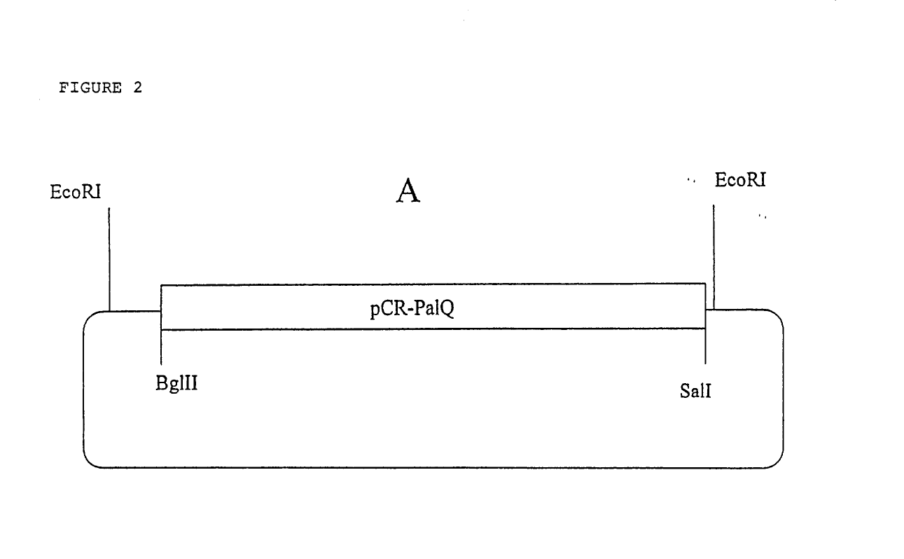

Primer FB 180 comprises the bases 2-21, primer FB 176 comprises the bases 1638-1656 of the coding region of the palatinase gene. For cloning the DNA into expression vectors the primers also have the following restriction sites: primer FB 180 BglII; primer FBI 76 SalI. The PCR reaction mix (100 μl) contained bacterial chromosomal DNA (1 μg), primer FBI 80 and FB 176 (250 ng in each case), Pfu DNA polymerase reaction buffer (10 μl, Stratagene), 200 μM dNTPs (dATP, dCTP, dGTP, dTTP) and 2.5 units of Pfu DNA polymerase (Stratagene). Before initiating the amplification cycles, the mixture was heated for 5 min to 95° C. The polymerisation steps (30 cycles) were carried out in an automated T3-Thermocycler (Biometra) according to the following program: denaturation at 95° C. (1 minute), annealing of the primers at 55° C. (40 seconds), polymerase reaction at 72° C. (2 minutes). The corresponding fragment was cloned into the vector pCR-Blunt (Invitrogen), resulting in the plasmid pCR-PalQ (FIG. 2). The identity of the amplified DNA was verified by sequence analysis. The fragment A contains the sequence of a palatinase from E. rhapontici, which extends from nucleotide 2-1656 of the palatinase gene (see SEQ ID NO: 1).

### Example 5

Production of plasmid p35S-cwIso

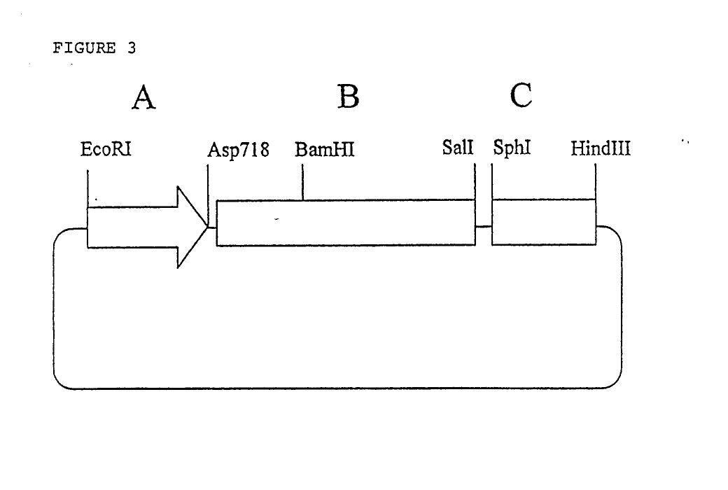

A DNA sequence which codes for a sucrose isomerase was isolated from the plasmid pCR-SucIso2 and was linked to the 35S promoter of the Cauliflower Mosaic Virus, which mediates a constitutive expression in transgenic plant cells, a leader peptide of a plant gene necessary for the transport (uptake) into the endoplasmic reticulum (proteinase-inhibitor II gene from potato (Keil et al. (1986) Nucl. Acids Res. 14:5641-5650; Gene bank Accession No. X04118), and a plant termination signal. For this purpose the sucrose isomerase fragment was cut out from the pCR-SucIso2 construct (see FIG. 1) by digestion via the restriction sites BamHI and SalI and ligated in a BamHI/SalI linearised pMA vector. The vector pMA is a modified form of the vector pBinAR (Höfgen and Willmitzer (1990) Plant Sci. 66:221-230) which contains the 35S promoter of the Cauliflower Mosaic Virus, which mediates a constitutive expression in transgenic plants, a leader peptide of the proteinase inhibitor II from potato which mediates the target control of the fusion protein into the cell wall, and a plant termination signal. The plant termination signal contains the 3′ end of the polyadenylation site of the octopine synthase gene. Between the subsequence of the proteinase inhibitor and the termination signal are specific sites for the restriction enzymes BamHI, XbaI, SalI, PstI and SphI (in this order), which allow the insertion of corresponding DNA fragments so that a fusion protein is created between the proteinase inhibitor and the introduced protein which is then transported into the cell wall of transgenic plant cells which express this protein (FIG. 3).

Fragment A contains the 35S promoter of the Cauliflower Mosaic Virus (CaMV). It contains one fragment which comprises the nucleotides 6909 or 7437 of the CaMV (Franck (1980) Cell 21:285.

Fragment B contains the nucleotides 923-1059 of a proteinase inhibitor II gene from potato (Keil et al., supra), which is fused via a linker with the sequence ACC GAA TTG GG (SEQ ID NO:16) to the sucrose isomerase gene from Erwinia rhapontici, which comprises the nucleotides 109-1803. By this means a leader peptide of a plant protein necessary for the transport of proteins into the endoplasmic reticulum (ER) is N-terminally fused to the sucrose isomerase sequence.

Fragment C contains the polyadenylation signal of gene 3 of the T-DNA of the Ti plasmid pTiACH5 (Gielen et al. (1984) EMBO J. 3:835), nucleotides 11749-11939.

In p35S-cwIso (35S=35S promoter, cw=cell wall, Iso=sucrose isomerase) the coding region of the sucrose isomerase from E. rhapontici is under constitutive control and the gene product is transported into the ER via uptake.

### Example 6

Production of Plasmid pTA29-cwIso

In a manner similar to that described in Example 5, the plasmid pTA29-cwIso was produced, but with the variation that the expression of the fusion protein from proteinase inhibitor leader peptide and the sucrose isomerase is brought under the control of the anther-specific promoter TA29 from tobacco. The functionality of the anther-specific TA29 promoter has already been demonstrated (Mariani et al. (1990) Nature 347:727-741). The plant termination signal contains the 3′ end of the polyadenylation site of the octopine synthase gene. The plasmid pTA29-cwIso contains three fragments A, B and C, which were cloned into the restriction sites for restriction enzymes of the polylinker of pUC18 (see FIG. 3).

Fragment A contains the TA29 promoter from Nicotiana tabacum. The fragment contains the nucleotides −1477 to +57 relative to the transcription initiation site of the TA29 gene (Seurinck et al. (1990) Nucl. Acids. Res. 18:3403). It was amplified by means of PCR from genomic DNA of Nicotiana tabacum Var. Samsun NN. The amplification was carried out using the specific primers:

| | |
|---|---|
| FB158 | |
| 5′-GAATTCGTTTGACAGCTTATCATCGAT-3′ | (SEQ ID NO:17) |
| and | |
| FB159 | |
| 5′-GGTACCAGCTAATTTCTTTAAGTAAA-3′. | (SEQ ID NO:18) |

For cloning the DNA into the expression cassette the primers also have the following restriction sites: primer FB 158, EcoRI; primer FB 159, Asp718. The PCR reaction mix (100 μl) contained genomic DNA of tobacco (2 μg), primers FB158 and FB159 (250 ng in each case), Pfu DNA polymerase reaction buffer (10 μl, Stratagene), 200 μM dNTPs (dATP, dCTP, dGTP, dTTP) and 2.5 units of Pfu DNA-polymerase (Stratagene). Before initiating the amplification cycles the mixture was heated for 5 min to 95° C. The polymerisation steps (35 cycles) were carried out in an automated T3-Thermocycler (Biometra) according to the following program: denaturation at 95° C. (1 minute), annealing of the primers at 55° C. (40 seconds), polymerase reaction at 72° C. (2 minutes). The amplicon was digested with the restriction enzymes EcoRA and Asp718 and cloned into the corresponding restriction sites of the polylinker of pUC18. The identity of the amplified DNA was verified by sequence analysis.

Fragment B contains the nucleotides 923 to 1059 of a proteinase inhibitor II gene from potato (Keil et al. (1986) Nucl. Acids Res. 14:5641-5650; Gene bank Accession No. X04118) which are fused via a linker with the sequence ACC GAA TTG GG (SEQ ID NO:16) to the sucrose isomerase gene from E. rhapontici, which comprises the nucleotides 109 to 1803. By this means a leader peptide of a plant protein required for the transport of proteins into the ER is N-terminally fused to the sucrose isomerase sequence. The fragment B was cut out as an Asp718/SalI fragment from the p35S-cwIso construct as described above (Example 5) and cloned between the restriction sites Asp718 and SalI of the polylinker region of pUC18.

Fragment C contains the polyadenylation signal of gene 3 of the T-DNA of the Ti plasmid pTiACH5 (Gielen et al. (1984) EMBO J. 3:835), nucleotides 11749-11939, which was isolated as a PvuII/HindIII fragment from the plasmid pAGV 40 (Herrera-Estrella et al. (1983) Nature 303:209) and has been cloned after addition of SphI linkers to the PvuII site between the SphI/HindIII-site of the polylinker of pUC18.

The chimeric gene was then cloned as a EcoRI/HindIII fragment between the EcoRI- and HindIII-site of the plasmid pBIN19 (Bevan (1984) Nucl. Acids Res. 12:8711).

In pTA29-cwIso (TA29=promoter of the TA29 gene from tobacco, cw=cell wall, Iso=sucrose isomerase) the coding region of the sucrose isomerase gene from E. rhapontici is under anther-specific control, the gene product is transported into ER via uptake.

Tobacco plant cells were transformed as described above with the construct pTA29-cwIso by means of agrobacterium-mediated gene transfer and whole tobacco plants were regenerated. The resulting pTA29-cwIso transformants showed a male sterile phenotype, otherwise there were no differences in their phenotype compared to the wild-type.

### Example 7

Production of Plasmid pTA29-cwPalQ

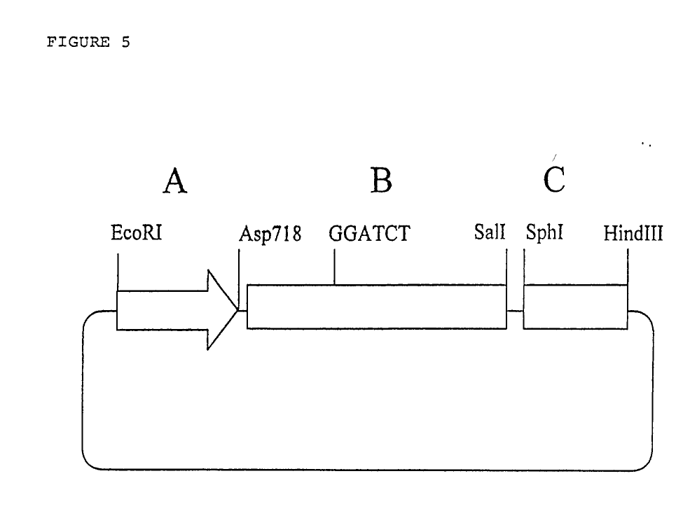

In a manner similar to that described in Example 6, the coding region of the palatinase from E. rhapontici was fused to a leader peptide of a plant gene necessary for the transport into the ER (proteinase inhibitor II gene from potato, Keil et al. (1986) vide supra) under the control of the anther-specific promoter of the TA29 gene from tobacco. The resulting construct pTA29-cwPalQ consists of three fragments A, B and C (seen in FIG. 5) and allows the expression of the palatinase in the cell wall of tapetum cells.

Fragment A contains the TA29 promoter from Nicotiana tabacum. The fragment contains the nucleotides −1477 to +57 relative to the transcription initiation site of the TA29 gene (Seurinck et al. (1990) Nucl. Acids. Res. 18:3403). It was amplified by means of PCR from genomic DNA of Nicotiana tabacum Var. Samsun NN. The amplification was carried out using the specific primers:

| | |
|---|---|
| FB158 | |
| 5′-GAATTCGTTTGACAGCTTATCATCGAT-3′ | (SEQ ID NO:17) |
| and | |
| FB159 | |
| 5′-GGTACCAGCTAATTTCTTTAAGTAAA-3′. | (SEQ ID NO:18) |

For cloning the DNA into the expression cassette the primers also have the following restriction sites: primer FB158, EcoRI; primer FB159, Asp718. The PCR reaction mix (100 μl) contained genomic DNA of tobacco (2 μg), primers FB158 and FB159 (250 ng in each case), Pfu DNA polymerase reaction buffer (10 μl, Stratagene), 200 μM dNTPs (dATP, dCTP, dGTP, dTTP) and 2.5 units of Pfu DNA-polymerase (Stratagene). Prior to the initiation of the amplification cycles the mixture was heated for 5 min to 95° C. The polymerisation steps (35 cycles) were carried out in an automated T3-Thermocycler (Biometra) according to the following program: denaturation at 95° C. (1 minute), annealing of the primers at 55° C. (40 seconds), polymerase reaction at 72° C. (2 minutes). The amplicon was digested with the restriction enzymes EcoRA and Asp718 and cloned into the corresponding restriction sites of the polylinker of pUC18. The identity of the amplified DNA was verified by sequence analysis.

Fragment B contains the nucleotides 923-1059 of a proteinase inhibitor II gene from potato (Solanum tuberosum, Keil et al. 1986, vide supra) which are fused via a linker with the sequence ACC GAA TTG GG (SEQ ID NO: 16) to the palatinase gene from Erwinia rhapontici, which comprises the nucleotides 2-1656. By this means a leader peptide of a plant protein required for the transport of proteins into the endoplasmic reticulum is N-terminally fused to the palatinase sequence.

For cloning fragment B the region of the proteinase inhibitor II gene comprising the nucleotides 923 to 1059 was isolated via the restriction enzymes Asp718 and BamHI from the pMA vector and cloned between the corresponding sites of the polylinker of pUC18. Finally, the palatinase fragment cut out from the pCR-PalQ construct via BglII and SalI was fused to the sequence of the proteinase inhibitor via the BamHI site compatible to the BglII site.

Fragment C contains the polyadenylation signal of gene 3 of the T-DNA of the Ti plasmid pTiACH5 (Gielen et al. (1984) EMBO J. 3:835), nucleotides 11749-11939, which was isolated as a PvuII/HindIII fragment from the plasmid pAGV40 (Herrera-Estrella et al. (1983) Nature 303:209) and has been cloned after adding SphI linkers to the PvuII site between the SphI- and HindIII-sites of the polylinker of pUC18. The chimeric gene was then cloned as a EcoRI/HindIII fragment between the EcoRI- and HindIII-sites of the plasmid pBIN19 (Bevan (1984) Nucl. Acids Res. 12:8711).

In pTA29-cwPalQ (TA29=promoter of the TA29 gene from tobacco, cw=cell wall, PalQ−palatinase) the coding region of the palatinase gene from E. rhapontici is under anther-specific control, the gene product is transported into the ER.

Transgenic plants, which were transformed with pTA29-cwPalQ by means of agrobacterium-mediated gene transfer, showed no difference in their phenotype compared to the wild-type. The daughter plants obtained from crossing these plants with the male sterile plants from Example 6 again showed the male fertile phenotype of the pTA29-cwPalQ parent plants.

### Example 8

Production of plasmid p35S-cwPalQ

The DNA sequence which codes for a palatinase was fused to a leader peptide of a plant gene necessary for the transport into the endoplasmic reticulum (proteinase-inhibitor II gene from potato (Solanum tuberosum, Keil et al. (1986, vide supra)) and was brought under control of the 35S RNA promoter, resulting in the constructed plasmid p35S-cwpalQ.

For this purpose the palatinase fragment was cut out from the pCR-palQ construct via the restriction sites BglII and SalI and ligated in a BamHI/SalI linearised pMA vector. The vector pMA is a modified form of the vector pBinAR (Höfgen and Willmitzer (1990) Plant Sci. 66:221-230) which contains the 35S promoter of the Cauliflower Mosaic Virus, which mediates a constitutive expression in transgenic plants, a leader peptide of the proteinase inhibitor II from potato (Keil et al. 1986, vide supra) which mediates the target control of the fusion protein into the cell wall, and a plant termination signal. The plant termination signal contains the 3′ end of the polyadenylation site of the octopine synthase gene. Between the partial sequence of the proteinase inhibitor and the termination signal are specific sites for the restriction enzymes BamHI, XbaI, SalI, PstI and SphI (in this order), which allow the insertion of corresponding DNA fragments so that a fusion protein is created between the proteinase inhibitor and the introduced protein which is then transported into the cell wall of transgenic plants or plant cells which express this protein (see FIG. 5).

Fragment A contains the 35S RNA promoter of the Cauliflower Mosaic Virus (CaMV). It contains one fragment which comprises the nucleotides 6909 to 7437 of the CaMV (Franck (1980) Cell 21:285).

Fragment B contains the nucleotides 923-1059 of a proteinase inhibitor II gene from potato (Solanum tuberosum, Keil et al. 1986, vide supra), which is fused via a linker having the sequence ACC GAA TTG GG to the palatinase gene from Erwinia rhapontici, which comprises the nucleotides 2-1656. By this means a leader peptide of a plant protein necessary for the transport of proteins into the endoplasmic reticulum (ER) is N-terminally fused to the palatinase sequence.

Fragment C contains the polyadenylation signal of gene 3 of the T-DNA of the Ti plasmid pTiACH5 (Gielen et al. (1984) EMBO J. 3:835), nucleotides 11749-11939.

In p35S-cwPalQ (35S=35S RNA promoter of the CaMV, cw=cell wall, PalQ=palatinase) the coding region of the palatinase gene from E. rhapontici is under constitutive control and the gene product is transported into the ER.

Transgenic plants, which were transformed with p35S-cwPal by means of agrobacterium-mediated gene transfer, showed no difference in their phenotype compared to the wild-type. The daughter plants obtained from crossing these plants with the male sterile plants from Example 6 again showed the male fertile phenotype of the p35S-cwPal parent plants.

### Example 9

Production of the Plasmid pMAL-SucIso

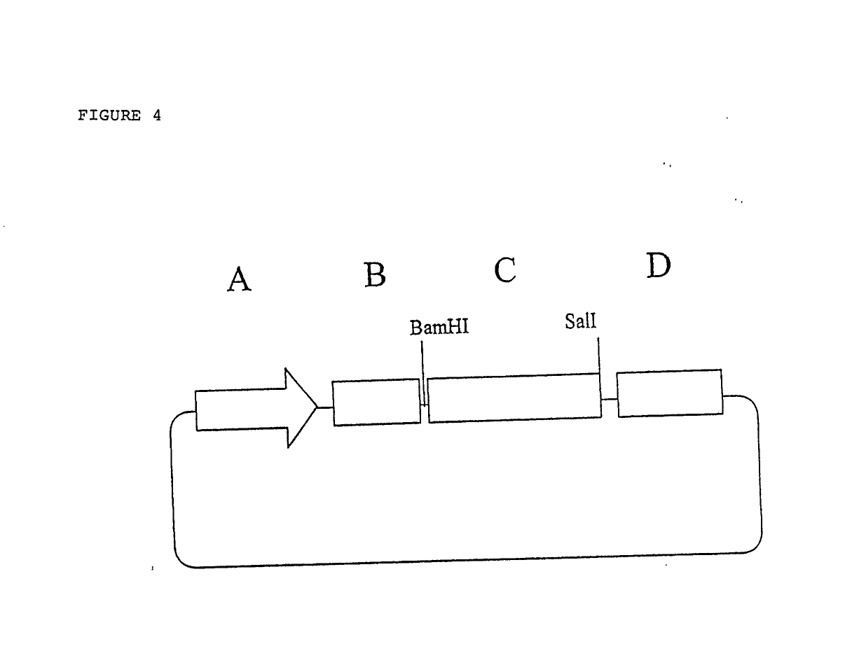

To produce the plasmid pMAL-SucIso the sucrose isomerase fragment was cut out from the construct pCR-SucIso2 via the restriction enzymes BamHI and SalI and ligated in a pMAL-c2 vector (New England Biolabs), which was also cut in this manner to create the construct pMAL-SucIso (FIG. 4). This allows an expression of the enzyme as fusion protein with the maltose-binding protein under control of the IPTG-inducible tac-promoter.

Fragment A contains the tac-promoter that allows IPTG-inducible gene expression.

Fragment B contains a region of the malE gene and the initiation of translation.

Fragment C contains the coding region of the sucrose isomerase.

Fragment D contains the rrnB-terminator from E. coli.

Bacterial cells transformed with pMAL-SucIso show IPTG-inducible expression of the sucrose isomerase from E. rhapontici.

### Example 10

Functional Detection of Sucrose Isomerase Activity in E. coli

Functional characterisation of the sucrose isomerase gene was implemented by expression in E. coli. For this purpose the plasmid pMAL-Suclso was transformed in E. coli (XL-I blue, Stratagene). The expression of the fusion protein between the maltose-binding protein and the sucrose isomerase was carried out according to the manufacturer's data on a 50 ml culture scale. After harvesting the cells the pellet was resuspended in 1 ml 50 mM sodium phosphate buffer (pH 6.0) and the soluble protein fraction was released by ultrasonication. An aliquot of the raw extract was mixed with the same volume of 600 mM sucrose and incubated for 24 hours at 30° C. An aliquot of the mixture was subjected to a HPLC analysis to detect the palatinose produced. The chromatogram confirmed the production of palatinose by detecting the recombinant sucrose isomerase in E. coli.

### Example 11

In vivo Detection of the Sucrose Isomerase Activity in Transgenic Plants

The in vivo functionality of the sucrose isomerase in transgenic plants was detected as follows: ethanol extracts were produced from 0.5 cm² leaf disks of untransformed tobacco plants and the transformants 35S-cwIso (from Example 5) and were analysed by HPLC, and the sugars were identified using the corresponding standards. As the chromatograms showed, the expression of the sucrose isomerase in the cell wall resulted in a substantial accumulation of palatinose in the analysed p35S-cwIso plants. The wild-type contains no palatinose, as also could be seen clearly from the chromatograms.

### Example 12

Functional Detection and Biochemical Characterisation of the Palatinase Activity in E. coli

The functional characterisation of the palatinase gene was implemented by expression of the recombinant protein in E. coli. For this purpose the plasmid pQE-palQ was transformed in E. coli (XL-I blue, Stratagene). The expression of the recombinant protein was carried out according to the manufacturer's data (Qiagen, Hilden, Germany) on a 50 ml culture scale. After harvesting the cells by centrifugation the pellet was resuspended in 1 ml 30 mM HEPES (pH 7.5) and the soluble protein fraction was released by ultrasonication. 20 μl of the raw extract were mixed with 80 μl of 100 mM palatinose and incubated for 40 minutes at 30° C. In order to detect the palatinase activity the released glucose was determined in an aliquot of the mixture by a coupled optical enzymatic test. Thus, the palatinase activity of the recombinant enzyme could be clearly detected. In further experiments it was demonstrated that the enzyme evolves its highest activity at a reaction temperature of 30° C. and a pH of 7.0. When the reaction rate was analysed depending on the concentration of the substrate, a Kₘ value of 10 mM for palatinose and a maximum reaction rate at a substrate concentration of 90 mM palatinose could be determined.

### Example 13

PCR-Amplification of a Trehalulase from Erwinia rhapontici

The entire open reading frame of trehalulase was cloned by means of polymerase chain reaction (Polymerase Chain Reaction, PCR). The template material was genomic DNA from E. rhapontici, which was isolated according to a standard protocol. The amplification was carried out using the specific primers:

| | |
|---|---|
| FB184 | |
| 5′-GGGATCCGTGCAAACTGGTGGAAAGAG-3′ | (SEQ ID NO:19) |
| FB185 | |
| 5′-GTCGACTTACCGCTGATAAATTTGTGC-3′ | (SEQ ID NO:20) |

The primers FB184 and FB185 comprise the bases 4-23 and 1638-1659, respectively, of the coding region of the trehalulase gene.

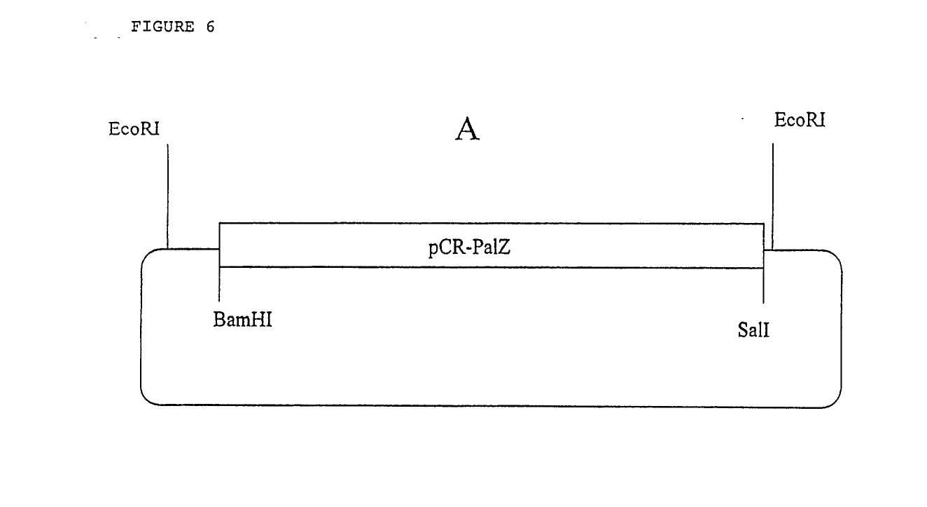

For cloning the DNA into expression vectors the primers additionally contain the following restriction sites: primer FB96 or FB 184: BamHI; primer FB 185: SalI. The PCR reaction mix (100 μl) contained bacterial chromosomal DNA (1 μg), primers FB184 and FB185 (250 ng in each case), Pfu DNA polymerase reaction buffer (10 μL, Stratagene), 200 μM dNTPs (dATP, dCTP, dGTP, dTTP) and 2.5 units Pfu DNA polymerase (Stratagene). Prior to the initiation of the amplification cycles the mixture was heated for 5 minutes to 95° C. The polymerisation steps (30 cycles) were carried out in an automated T3-Thermocycler (Biometra) according to the following program: denaturation 95° C. (1 minute), annealing of the primers at 55° C. (40 seconds), polymerase reaction at 72° C. (2 minutes). The amplicon was digested with BamHI and SalI and the fragment was cloned into the vector pCR-Blunt (Invitrogen), which resulted in the plasmid pCR-PalZ (see FIG. 6). The identity of the amplified DNA was verified by means of sequence analysis.

Fragment A contains the sequence of a trehalulase from E. rhapontici, which extends from nucleotide 4-1659 of the trehalulase gene.

### Example 14

Production of the Plasmid pTA29-cwPalZ

In a procedure similar to that described in Example 7, the coding region of the trehalulase gene from Erwinia rhapontici was fused to a leader peptide of a plant gene necessary for the transport into the endoplasmic reticulum (proteinase inhibitor II gene from potato (Solanum tuberosum, Keil et al. (1986) vide supra) under the control of the anther-specific promoter of the TA29 gene from tobacco. The so obtained construct pTA29-cwPalZ consists of three fragments A, B and C (see FIG. 3) and allows the expression of the trehalulase in the cell wall of tapetum cells.

Fragment A contains the TA29 promoter from Nicotiana tabacum. The fragment contains the nucleotides −1477 to +57 relative to the initiation of transcription of the TA29 gene (Seurinck et al. (1990) Nucleic Acids Res. 18:3403). It was amplified by means of PCR from genomic DNA of Nicotiana tabacum Var. Samsun NN. The amplification was carried out using the specific primers:

| | |
|---|---|
| FB158 | |
| 5′-GAATTCGTTTGACAGCTTATCATCGAT-3′ | (SEQ ID NO:17) |
| and | |
| FB159 | |
| 5′-GGTACCAGCTAATTTCTTTAAGTAAA-3′. | (SEQ ID NO:18) |

For cloning the DNA into the expression cassette the primers also have the following restriction sites: primer FB 158, EcoRI; primer FB 159, Asp718. The PCR reaction mix (100 μl) contained genomic DNA of tobacco (2 μg), primers FB158 and FB159 (250 ng in each case), Pfu DNA polymerase reaction buffer (10 μl, Stratagene), 200 μM dNTPs (dATP, dCTP, dGTP, dTTP) and 2.5 units of Pfu DNA polymerase (Stratagene). Prior to the initiation of the amplification cycles the mixture was heated for 5 min to 95° C. The polymerisation steps (35 cycles) were carried out in an automated T3-Thermocycler (Biometra) according to the following program: denaturation at 95° C. (1 minute), annealing of the primers at 55° C. (40 seconds), polymerase reaction at 72° C. (2 minutes). The amplicon was digested with the restriction enzymes EcoRI and Asp718 and ligated into the corresponding sites of the polylinker of pUC18. The identity of the amplified DNA was verified by means of sequence analysis.

Fragment B contains the nucleotides 923-1059 of a proteinase inhibitor II gene from potato (Solanum tuberosum, Keil et al. (1986), vide supra), which is fused via a linker with the sequence ACC GAA TTG GG to the trehalulase gene from Erwinia rhapontici, which comprises the nucleotides 4-1659. By this means a leader peptide of a plant protein required for the transport of proteins into the endoplasmic reticulum is N-terminally fused to the trehalulase sequence.

Fragment C contains the polyadenylation signal of gene 3 of the T-DNA of the Ti plasmid pTiACH5 (Gielen et al. (1984) EMBO J. 3:835), nucleotides 11749-11939, which was isolated as a PvuII/HindIII fragment from the plasmid pAGV 40 (Herrera-Estrella et al. (1983) Nature 303, 209) and has been cloned after addition of SphI linkers to the PvuII site between the SphI- and HindIII-sites of the polylinker of pUC18.

The chimeric gene was then cloned as a EcoRI/HindIII fragment between the EcoRI/HindIII sites of the plasmid pBIN19 (Bevan (1984) Nucleic Acids Res. 12, 8711).

In pTA29-cwPalZ (TA29=promoter of the TA29 gene from tobacco, cw=cell wall, PalZ−trehalulase) the coding region of the trehalulase gene from E. rhapontici is under anther-specific control, the gene product is transported into the ER.

Transgenic plants, which were transformed with pTA29-cwPalZ by means of agrobacterium-mediated gene transfer, showed no difference in their phenotype compared to the wild-type. The daughter plants obtained from crossing these plants with the male sterile plants from Example 6 again showed the male fertile phenotype of the pTA29-cwPalZ parent plants.

### Example 15

Site-Directed Mutagenesis of the Palatinase from E. rhapontici to Optimise Palatinase Expression in Transgenic Plants

In order to avoid glycosylation of the palatinase from E rhapontici in transgenic plants, suitable amino acids were substituted by site-directed mutagenesis in the region of potential glycosylation sites. The plasmid pQE-palQ was used as template. As far as the palatinase sequence is concerned, pQE-palQ corresponds to pCR-palQ, but is suitable for the expression of the palatinase sequence in E. coli. The reaction mixture (50 μl) for PCR-supported mutagenesis was composed as follows: 50 ng pQE-palQ DNA, 250 ng each of 5′ or 3′ primer, Pfu DNA polymerase reaction buffer (5 μl, Stratagene), 200 μM dNTPs (dATP, dCTP, dGTP, dTTP) and 2.5 units of Pfu-DNA-polymerase (Stratagene). The polymerisation steps (15 cycles) were carried out in an automated T3-Thermocycler (Biometra) according to the following program: denaturation at 95° C. (30 seconds), annealing of the primers at 55° C. (1 minute), polymerase reaction at 72° C. (15 minutes). After completion of the reaction the parental DNA was digested with 1 unit of restriction enzyme DpnI for 1 hour at 37° C. Then 1 μl of the mixture was used for the transformation of E. coli.

For mutation 1 threonine at position 105 was substituted by alanine, thereby resulting in plasmid pQE-palQ T105A.

| | | |
|---|---|---|
| 5′-Primer: SL36 | 5′-CTG GTG GTC AAC CAT GCC TCT GAC GAA CAT CCC-3′ | (SEQ ID NO:21) |
| 3′-Primer: SL37 | 5′-GGG ATG TTC GTC AGA GGC ATG GTT GAC CAC CAG-3′ | (SEQ ID NO:22) |

For mutation 2 threonine at position 248 was substituted by alanine, thus resulting in plasmid pQE-palQ T248A.

| | | |
|---|---|---|
| 5′-Primer: SL39 | 5′-GAG ACG TGG AGC GCA GCG CCA GAA GAC GCC CTG-3′ | (SEQ ID NO:23) |
| 3′-Primer: SL40 | 5′-CAG GGC GTC TTC TGG CGC TGC GCT CCA CGT CTC-3′ | (SEQ ID NO:24) |

For mutation 3 threonine at position 502 was substituted by alanine, resulting in plasmid pQE-palQ T502A.

| | | |
|---|---|---|
| 5′-Primer: SL42 | 5′-G GTG ATC AAT AAC TTC GCG CGA GAC GCT GTG ATG C-3′ | (SEQ ID NO:25) |
| 3′-Primer: SL43 | 5′-G CAT CAC AGC GTC TCG CGC GAA GTT ATT GAT CAC C-3′ | (SEQ ID NO:26) |

The mutation event was in each case verified by sequencing the corresponding region of the palatinase sequence. Functional expression of the mutagenised enzyme in E. coli could demonstrate in all cases that the respective amino acid substitution does not have any disadvantageous effect on the enzymatic activity. The mutations were then linked to each other by the above-mentioned strategy so that a palatinase was finally produced which has no putative glycosylation sites left. After expression in E. coli also this enzyme showed no disadvantageous catalytic properties.

| | | |
|---|---|---|
| (a) palQ wild-type | 100 105 110 | |
| amino acid sequence | Leu Val Val Asn His Thr Ser Asp Glu His Pro | (SEQ ID NO:27) |
| nucleotide sequence | CTG GTG GTC AAC CAT ACC TCT GAC GAA CAT CCC | (SEQ ID NO:28) |
| (b) palQ T105A | | |
| amino acid sequence | • • • • • Ala • • • • • | (SEQ ID NO:29) |
| nucleotide sequence | ... ... ... ... ... GCC ... ... ... ... ... | (SEQ ID NO:30) |
| (c) palQ wild-type | 243 248 253 | |
| amino acid sequence | Glu Thr Trp Ser Ala Thr Pro Glu Asp Ala Leu | (SEQ ID NO:31) |
| nucleotide sequence | GAG ACG TGG AGC GCA ACG CCA GAA GAC GCC CTG | (SEQ ID NO:32) |
| (d) palQ T248A | | |
| amino acid sequence | • • • • • Ala • • • • • | (SEQ ID NO:33) |
| nucleotide sequence | ... ... ... ... ... GCC ... ... ... ... ... | (SEQ ID NO:34) |
| (e) palQ wild-type | 497 502 507 | |
| amino acid sequence | Val Ile Asn Asn Phe Thr Arg Asp Ala Val Met | (SEQ ID NO:35) |
| nucleotide sequence | GTG ATC AAT AAC TTC ACG CGA GAC GCT GTG ATG | (SEQ ID NO:36) |
| (f) palQ T502A | | |
| amino acid sequence | • • • • • Ala • • • • • | (SEQ ID NO:37) |
| nucleotide sequence | ... ... ... ... ... GCC ... ... ... ... ... | (SEQ ID NO:38) |

The mutated palatinase sequence was subsequently subcloned into a plant transformation vector and was expressed in plants.

What is claimed is:

1. A method for influencing pollen development in transgenic plants by modifying the carbohydrate metabolism, comprising the following steps:
a) producing a recombinant nucleic acid molecule, comprising the following sequences:
regulatory sequences of a promoter that is active in anthers, in the tapetum and/or in pollen;
operatively linked thereto a DNA sequence which codes for a protein having the enzymatic activity of a sucrose isomerase; and
operatively linked thereto regulatory sequences which can serve as transcription, termination and/or polyadenylation signals in plant cells;
b) transferring the nucleic acid molecule from a) to plant cells and
c) regenerating transgenic plants.

2. The recombinant nucleic acid molecule, comprising
a) regulatory sequences of a promoter that is active in anthers, in the tapetum and/or in pollen;
b) operatively linked thereto a DNA sequence which codes for a protein having the enzymatic activity of a sucrose isomerase; and
c) operatively linked thereto regulatory sequences which can serve as transcription, termination and/or polyadenylation signals in plant cells.

3. The recombinant nucleic acid molecule according to claim 2, wherein the DNA sequence originates from Erwinia rhapontici.

4. The recombinant nucleic acid molecule according to claim 2, wherein the promoter is the T29 promoter.

5. A vector comprising a recombinant nucleic acid molecule according to claim 2.

6. A microorganism comprising a recombinant nucleic acid molecule according to any one of claims 2 to 4.

7. A microorganism comprising the vector of claim 5.

8. A method for generating male sterile plants comprising the transfer of a recombinant nucleic acid molecule according to any one of claims 2 to 4 to plant cells.

9. A method for generating male sterile plants comprising the transfer of the vector of claim 5 to plant cells.

10. A transgenic plant cell comprising a recombinant nucleic acid molecule according to any one of claims 2 to 4.

11. A transgenic plant cell comprising the vector of claim 5.

12. A transgenic plant, protoplast, callus, seed, tuber, cutting, or harvest product comprising the plant cell of claim 11.

13. A recombinant nucleic acid molecule comprising a DNA sequence from Erwinia rhapontici or a part thereof that codes for a protein having the enzymatic activity of a palatinase.

14. A recombinant nucleic acid molecule according to claim 13, wherein the DNA sequence is SEQ ID No. 1.

15. A recombinant nucleic acid molecule containing a DNA sequence from Erwinia rhapontici or a part thereof that codes for a protein having the enzymatic activity of a trehalulase.

16. The recombinant nucleic acid molecule according to claim 15, wherein the DNA sequence is SEQ ID No. 7.

17. A method for generating male fertile hybrid plants, comprising the following steps:
a) producing a first transgenic male sterile parent plant, comprising a nucleic acid molecule which codes for a protein having the enzymatic activity of a sucrose isomerase,
b) producing a second transgenic parent plant, comprising a nucleic acid molecule which codes for a protein having the enzymatic activity of a palatinase and/or a protein having the enzymatic activity of a trehalulase,
c) crossing the first parent plant with the second parent plant to generate a hybrid plant,
wherein the hybrid plant is male fertile.

18. A method for generating male fertile hybrid plants comprising the following steps:
a) producing a first transgenic male sterile parent plant comprising a nucleic acid molecule which codes for a protein having the enzymatic activity of a sucrose isomerase,
b) producing a second transgenic parent plant comprising a nucleic acid molecule which codes for a protein which has the biological activity of a sucrose isomerase inhibitor,
c) crossing the first parent plant with the second parent plant for generating a hybrid plant,
wherein the hybrid plant is male fertile.

19. A method for generating male fertile hybrid plants comprising the following steps:
a) producing a first transgenic male sterile parent plant comprising a nucleic acid molecule which codes for a protein having the enzymatic activity of a sucrose isomerase,
b) producing a second transgenic parent plant comprising a nucleic acid molecule which codes for a ribozyme which is directed against sucrose isomerase mRNA,
c) crossing the first parent plant with the second parent plant for generating a hybrid plant,
wherein the hybrid plant is male fertile.

20. A method for generating male fertile hybrid plants, comprising the following steps:
a) producing a first transgenic male sterile parent plant comprising a nucleic acid molecule which codes for a protein having the enzymatic activity of a sucrose isomerase,
b) producing a second transgenic parent plant comprising a nucleic acid molecule which codes for a sucrose isomerase antisense or sense RNA,
c) crossing the first parent plant with the second parent plant to generate a hybrid plant,
wherein the hybrid plant is male fertile.

21. The method of claim 17, wherein a glycosylation site is inactivated within the protein having the enzymatic activity of a palatinase and/or the protein having the enzymatic activity of a trehalulase due to at least one amino acid exhange in comparison with the wild type protein.

22. The recombinant nucleic acid molecule of claim 13, wherein a glycosylation site is inactivated within the protein having the enzymatic activity of a palatinase due to at least one amino acid exchange in comparison with the wild type protein.


SEQUENCE LISTING (38 sequences)

SEQ ID NO: 1 - DNA, 1656 bp, Erwinia rhapontici
atgcgcagcacaccgcactggaaagaggccgtggtttatcaggtctatccgcgcagctttatggacagtaacggcgacggtaccggcgatctcaacggtattatcagcaagctcgattacctgcaacagctcggcatcacgctgttgtggctgtcgcccgtataccgttcgccgatggacgataacggctatgacatctctgattacgaagagattgccgacatttttggttcgatgagcgacatggagcgcctgattgcagaagctaaagcgcgtgatatcgggatcctgatggatctggtggtcaaccatacctctgacgaacatccctggtttatcgacgcactgagctcaaaaaacagtgcttaccgtgacttttatatctggcgagcaccggcggcagacggcgggccgcctgatgactctcgttcgaactttggtggcagtgcctggacgcttgatgaggccagcggtgaatactacctgcatcagttttccacgcgccagcccgatctcaactgggaaaacccgcgcgttcgtgaagccatccacgccatgatgaaccgctggctggataagggcatcgggggattccgaatggacgttatcgacctgatcgggaaagaagtggatccacagatcatggcgaatggtcgtcatcctcacctgtatcttcagcagatgaaccgggcgacctttggcccgcgcggcagcgtgacggtaggggagacgtggagcgcaacgccagaagacgccctgctctacagtgccgaagagcggcaagagcggcaagagctgacgatggtctttcagtttgagcacatcaaacttttctgggatgaacagtacgggaagtggtgtaaccagccgtttgatctgttgcgctttaaggccgtgattgacaagtggcagacggcactggctgaccatggctggaactcgttgttctggagcaaccatgatttgcctcgcgcggtctccaaatttggtgacgacggtgagtatcgcgtggtatcagcaaaaatgctcgccaccgcgcttcactgccttaaaggcacaccttacatttatcagggtgaagagattggcatgaccaacgtgaattttgctgatattgacgactatcgggatattgaaagcctgaatctttatcaggagcggatcgccgaagggatgagccacgaagcgatgatgcgcggtatccacgccaacgggcccgataatgcgcgaacgccaatgcagtggacagcagtccacatgccgggtttaccaccggtcagccctggattgaggctaatcctaacttcaggacagtggaatgtcgcggctgcgcttgacgatcctgactctgttttttaccactaccagaagctggtggcattgcgtaagcagctgccgctgctggtgcacggcgacttcaggcagatcgttgtcgaacatccgcaggtgtttgcctggctgcgcacgctgggggagcagacgctggtggtgatcaataacttcacgcgagacgctgtgatgctggcgatccccgacaatctgcagagccagcagggccgttgtctcatcaacaattacgcgccacgggagcagttggagccgattatggaactgcaaccttatgaatcctttgcattacttattgagaggctgtga

SEQ ID NO: 2 - DNA, 1656 bp, Erwinia rhapontici; identical to SEQ ID NO: 1
  CDS (1)..(1653)

SEQ ID NO: 3 - protein, 551 aa, Erwinia rhapontici
MRSTPHWKEAVVYQVYPRSFMDSNGDGTGDLNGIISKLDYLQQLGITLLWLSPVYRSPMDDNGYDISDYEEIADIFGSMSDMERLIAEAKARDIGILMDLVVNHTSDEHPWFIDALSSKNSAYRDFYIWRAPAADGGPPDDSRSNFGGSAWTLDEASGEYYLHQFSTRQPDLNWENPRVREAIHAMMNRWLDKGIGGFRMDVIDLIGKEVDPQIMANGRHPHLYLQQMNRATFGPRGSVTVGETWSATPEDALLYSAEERQERQELTMVFQFEHIKLFWDEQYGKWCNQPFDLLRFKAVIDKWQTALADHGWNSLFWSNHDLPRAVSKFGDDGEYRVVSAKMLATALHCLKGTPYIYQGEEIGMTNVNFADIDDYRDIESLNLYQERIAEGMSHEAMMRGIHANGPDNARTPMQWTAVHMPGLPPVSPGLRLILTSGQWNVAAALDDPDSVFYHYQKLVALRKQLPLLVHGDFRQIVVEHPQVFAWLRTLGEQTLVVINNFTRDAVMLAIPDNLQSQQGRCLINNYAPREQLEPIMELQPYESFALLIERL

SEQ ID NO: 4 - DNA, 1803 bp, Erwinia rhapontici
atgtcctctcaaggattgaaaacggctgtcgctatttttcttgcaaccactttttctgccacatcctatcaggcctgcagtgccgggccagataccgccccctcactcaccgttcagcaatcaaatgccctgcccacatggtggaagcaggctgttttttatcaggtatatccacgctcatttaaagatacgaatggggatggcattggggatttaaacggtattattgagaatttagactatctgaagaaactgggtattgatgcgatttggatcaatccacattacgattcgccgaatacggataatggttatgacatccgggattaccgtaagataatgaaagaatacggtacgatggaagactttgaccgtcttatttcagaaatgaagaaacgcaatatgcgtttgatgattgatattgttatcaaccacaccagcgatcagcatgcctggtttgttcagagcaaatcgggtaagaacaacccctacagggactattacttctggcgtgacggtaaggatggccatgcccccaataactatccctccttcttcggtggctcagcctgggaaaaagacgataaatcaggccagtattacctccattactttgccaaacagcaacccgacctcaactgggacaatcccaaagtccgtcaagacctgtatgacatgctccgcttctggttagataaaggcgtttctggtttacgctttgataccgttgccacctactcgaaaatcccgaacttccctgaccttagccaacagcagttaaaaaatttcgccgaggaatatactaaaggtcctaaaattcacgactacgtgaatgaaatgaacagagaagtattatcccactatgatatcgccactgcgggggaaatatttggggttcctctggataaatcgattaagtttttcgatcgccgtagaaatgaattaaatatagcgtttacgtttgatctgatcaggctcgatcgtgatgctgatgaaagatggcggcgaaaagactggaccctttcgcagttccgaaaaattgtcgataaggttgaccaaacggcaggagagtatgggtggaatgcctttttcttagacaatcacgacaatccccgcgcggtttctcactttggtgatgatcgaccacaatggcgcgagcatgcggcgaaagcactggcaacattgacgctgacccagcgtgcaacgccgtttatctatcagggttcagaactcggtatgaccaattatccctttaaaaaaatcgatgatttcgatgatgtagaggtgaaaggtttttggcaagactacgttgaaacaggcaaagtgaaagctgaggaattccttcaaaacgtacgccaaaccagccgtgataacagcagaacccccttccagtgggatgcaagcaaaaacgcgggctttaccagtggaaccccctggttaaaaatcaatcccaattataaagaaatcaacagcgcagatcagattaataatccaaattccgtatttaactattatagaaagctgattaacattcgccatgacatccctgccttgacctacggcagttatattgatttagaccctgacaacaattcagtctatgcttacacccgaacgctcggcgctgaaaaatatcttgtggtcattaattttaaagaagaagtgatgcactacaccctgcccggggatttatccatcaataaggtgattactgaaaacaacagtcacactattgtgaataaaaatgacaggcaactccgtcttgaaccctggcagtcgggcatttataaacttaatccgtag

SEQ ID NO: 5 - DNA, 1803 bp, Erwinia rhapontici; identical to SEQ ID NO: 4
  CDS (1)..(1800)

SEQ ID NO: 6 - protein, 600 aa, Erwinia rhapontici
MSSQGLKTAVAIFLATTFSATSYQACSAGPDTAPSLTVQQSNALPTWWKQAVFYQVYPRSFKDTNGDGIGDLNGIIENLDYLKKLGIDAIWINPHYDSPNTDNGYDIRDYRKIMKEYGTMEDFDRLISEMKKRNMRLMIDIVINHTSDQHAWFVQSKSGKNNPYRDYYFWRDGKDGHAPNNYPSFFGGSAWEKDDKSGQYYLHYFAKQQPDLNWDNPKVRQDLYDMLRFWLDKGVSGLRFDTVATYSKIPNFPDLSQQQLKNFAEEYTKGPKIHDYVNEMNREVLSHYDIATAGEIFGVPLDKSIKFFDRRRNELNIAFTFDLIRLDRDADERWRRKDWTLSQFRKIVDKVDQTAGEYGWNAFFLDNHDNPRAVSHFGDDRPQWREHAAKALATLTLTQRATPFIYQGSELGMTNYPFKKIDDFDDVEVKGFWQDYVETGKVKAEEFLQNVRQTSRDNSRTPFQWDASKNAGFTSGTPWLKINPNYKEINSADQINNPNSVFNYYRKLINIRHDIPALTYGSYIDLDPDNNSVYAYTRTLGAEKYLVVINFKEEVMHYTLPGDLSINKVITENNSHTIVNKNDRQLRLEPWQSGIYKLNP

SEQ ID NO: 7 - DNA, 1659 bp, Erwinia rhapontici
atggcaaactggtggaaagaggccgtggcgtatcagatatacccgcgcagcttcaacgacagcaataacgatggcattggtgacctgaatggcatcacggaaaaactcgattacctggaagatttgggcatcgatctgatttggatctgccccatgtatcagtcccccaatgatgacaacggctatgacatcagcgattaccagaaaatcatggctgagtttggcacgatggacgattttgaccgtctgcttgaacaggtgcatgcgcgcggtatgcgcctgattattgatttagtggtgaaccatacttctgatgagcatccgtggtttttagcgtccagcgcatcacgggataacccgaaacgcgactggtacatctggcgcgacggtaaagcgggcgctgagccgaacaactgggaaagcatcttcaacggttctgcctggaaatacagcgcggcgaccgggcagtatttcctgcatttgttctccgaaaagcagccagatttgaactgggaaaaccccgaggtgcgttcggcggtgtatgccatgatgcgttggtggcttgacaaaggggtagatggttttcgcattgatgccatctgccatatgaaaaaagagccgactttcagcgatatgcctaatcccctggcgctgccttacgtaccgtcattcgagcgccacctcaactacgacggcctgcttgattacgtcgatgacatgtgtgaacaggtgttcagtcactatgacattgtgaccatcggcgaaatgaacggtgcctccgctgaacagggtgaagagtgggtcggcgagcagcggggcaggctgaatatgatctttcagtttgagcacgtgaagctgtggcaggatgggcaaaaggacaccctggaggccagtctcgatttacccggcttaaaagagattttcacgcgctggcagacactgctggaaaacaaaggctggaacgcgttatacgtagaaaatcatgatcttccccgtgtggtatcgggctggggcgacgataaaaattatcaacgtgaaagcgcgaccgccattgcggcgatgttcttcctgatgaaaggtacgccgtttatttatcaggggcaggaacttggcatgaccaatacgcatttcgccagcctggaggattttgacgacgttgccgcgaagaaactcgccgttgaaatgcgccgacagggcagggaagagcccgagatcctcgccttcctcagccgcaccgggcgcgaaaactcgcgcaccccgatgcagtcggatcagagtgcccacggcggtttcagcaatgctaccccctggtttcctgcgaacagtaattaccctgtaatcaacgtggcggatcaacgtgctgacagcggttccgtgctgaacttctatcgtgcgctcattcgcctgcgccggcagatgccggtattgattgaaggggcttatcaacttctgctgccgacacatccgcagatctatgcctatacccgtcgtctgaatgaacagcaggtgttggtgatcgtcaatttcagtgcccatcagcaggagatagatccgcaacagctgttactggacggctggcaaccgctgctgagcaattatcaggagcaggggaaacggcaaatcttacgggcttatgaggcacaaatttatcagcggtaa

SEQ ID NO: 8 - DNA, 1659 bp, Erwinia rhapontici; identical to SEQ ID NO: 7
  CDS (1)..(1656)

SEQ ID NO: 9 - protein, 552 aa, Erwinia rhapontici
MANWWKEAVAYQIYPRSFNDSNNDGIGDLNGITEKLDYLEDLGIDLIWICPMYQSPNDDNGYDISDYQKIMAEFGTMDDFDRLLEQVHARGMRLIIDLVVNHTSDEHPWFLASSASRDNPKRDWYIWRDGKAGAEPNNWESIFNGSAWKYSAATGQYFLHLFSEKQPDLNWENPEVRSAVYAMMRWWLDKGVDGFRIDAICHMKKEPTFSDMPNPLALPYVPSFERHLNYDGLLDYVDDMCEQVFSHYDIVTIGEMNGASAEQGEEWVGEQRGRLNMIFQFEHVKLWQDGQKDTLEASLDLPGLKEIFTRWQTLLENKGWNALYVENHDLPRVVSGWGDDKNYQRESATAIAAMFFLMKGTPFIYQGQELGMTNTHFASLEDFDDVAAKKLAVEMRRQGREEPEILAFLSRTGRENSRTPMQSDQSAHGGFSNATPWFPANSNYPVINVADQRADSGSVLNFYRALIRLRRQMPVLIEGAYQLLLPTHPQIYAYTRRLNEQQVLVIVNFSAHQQEIDPQQLLLDGWQPLLSNYQEQGKRQILRAYEAQIYQR

SEQ ID NO: 10 - DNA, 27 bp, Erwinia rhapontici
ggatccggtaccgttcagcaatcaaat

SEQ ID NO: 11 - DNA, 23 bp, Erwinia rhapontici
gtcgacgtcttgccaaaaacctt

SEQ ID NO: 12 - DNA, 33 bp, Artificial Sequence
  A primer
ggatccacaatggcaaccgttcagcaatcaaat

SEQ ID NO: 13 - DNA, 25 bp, Erwinia rhapontici
gtcgacctacgtgattaagtttata

SEQ ID NO: 14 - DNA, 27 bp, Artificial Sequence
  A primer
gagatcttgcgcagcacaccgcactgg

SEQ ID NO: 15 - DNA, 24 bp, Artificial Sequence
  A primer
gtcgactcacagcctctcaataag

SEQ ID NO: 16 - DNA, 11 bp, Artificial Sequence
  A linker sequence
accgaattggg

SEQ ID NO: 17 - DNA, 27 bp, Nicotiana tabacum
gaattcgtttgacagcttatcatcgat

SEQ ID NO: 18 - DNA, 26 bp, Nicotiana tabacum
ggtaccagctaatttctttaagtaaa

SEQ ID NO: 19 - DNA, 27 bp, Artificial Sequence
  A primer
gggatccgtgcaaactggtggaaagag

SEQ ID NO: 20 - DNA, 27 bp, Artificial Sequence
  A primer
gtcgacttaccgctgataaatttgtgc

SEQ ID NO: 21 - DNA, 33 bp, Artificial Sequence
  A primer
ctggtggtcaaccatgcctctgacgaacatccc

SEQ ID NO: 22 - DNA, 33 bp, Artificial Sequence
  A primer
gggatgttcgtcagaggcatggttgaccaccag

SEQ ID NO: 23 - DNA, 33 bp, Artificial Sequence
  A primer
gagacgtggagcgcagcgccagaagacgccctg

SEQ ID NO: 24 - DNA, 33 bp, Artificial Sequence
  A primer
cagggcgtcttctggcgctgcgctccacgtctc

SEQ ID NO: 25 - DNA, 35 bp, Artificial Sequence
  A primer
ggtgatcaataacttcgcgcgagacgctgtgatgc

SEQ ID NO: 26 - DNA, 35 bp, Artificial Sequence
  A primer
gcatcacagcgtctcgcgcgaagttattgatcacc

SEQ ID NO: 27 - protein, 11 aa, Erwinia rhapontici
LVVNHTSDEHP

SEQ ID NO: 28 - DNA, 33 bp, Erwinia rhapontici
ctggtggtcaaccatacctctgacgaacatccc

SEQ ID NO: 29 - protein, 11 aa, Artificial Sequence
  Site-directed mutagenesis of palatinase from Erwinia rhapontici
LVVNHASDEHP

SEQ ID NO: 30 - DNA, 33 bp, Artificial Sequence; identical to SEQ ID NO: 21
  Site-directed mutagenesis of palatinase from Erwinia rhapontici

SEQ ID NO: 31 - protein, 11 aa, Erwinia rhapontici
ETWSATPEDAL

SEQ ID NO: 32 - DNA, 33 bp, Erwinia rhapontic
gagacgtggagcgcaacgccagaagacgccctg

SEQ ID NO: 33 - protein, 11 aa, Artificial Sequence
  Site-directed mutagenesis of palatinase from Erwinia rhapontici
ETWSAAPEDAL

SEQ ID NO: 34 - DNA, 33 bp, Artificial Sequence; identical to SEQ ID NO: 23
  Site-directed mutagenesis of palatinase from Erwinia rhapontici

SEQ ID NO: 35 - protein, 11 aa, Erwinia rhapontic
VINNFTRDAVM

SEQ ID NO: 36 - DNA, 33 bp, Erwinia rhapontic
gtgatcaataacttcacgcgagacgctgtgatg

SEQ ID NO: 37 - protein, 11 aa, Artificial Sequence
  Site-directed mutagenesis of palatinase from Erwinia rhapontici
VINNFARDAVM

SEQ ID NO: 38 - DNA, 33 bp, Artificial Sequence; identical to SEQ ID NO: 36
  Site-directed mutagenesis of palatinase from Erwinia rhapontici